SEQUENCE LISTING (16 sequences)

SEQ ID NO: 1 - DNA, 783 bp, Artificial Sequence
  Ovacyt polynucleotide
atgggctccatcggtgcagcaagcatggaattttgttttgatgtattcaaggagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaa

SEQ ID NO: 2 - DNA, 1161 bp, Artificial Sequence
  Ovasec polynucleotide
atgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatggaattatcagaaatgtccttcagccaagctccgtggattctcaaactgcaatggttctggttaatgccattgtcttcaaaggactgtgggagaaaacatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaa

SEQ ID NO: 3 - DNA, 3204 bp, Artificial Sequence
  Tgrn polynucleotide
atgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 4 - protein, 1067 aa, Artificial Sequence
  Tgrn amino acid
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 5 - DNA, 3204 bp, Artificial Sequence
  Tnrg polynucleotide
atggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgcatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgtga

SEQ ID NO: 6 - protein, 1067 aa, Artificial Sequence
  Tnrg amino acid
MVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 7 - DNA, 3204 bp, Artificial Sequence
  Tngr polynucleotide
atggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgcatgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgtaa

SEQ ID NO: 8 - protein, 1067 aa, Artificial Sequence
  Tngr amino acid
MVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 9 - DNA, 3204 bp, Artificial Sequence
  Trgn polynucleotide
atgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 10 - protein, 1067 aa, Artificial Sequence
  Trgn amino acid
MGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 11 - DNA, 3201 bp, Artificial Sequence
  Trng polynucleotide
atgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggccggagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgcatgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttg

SEQ ID NO: 12 - protein, 1067 aa, Artificial Sequence
  Trng amino acid
MGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCMGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVL

SEQ ID NO: 13 - DNA, 3204 bp, Artificial Sequence
  Tgnr polynucleotide
atgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgcatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgtaa

SEQ ID NO: 14 - protein, 1067 aa, Artificial Sequence
  Tgnr amino acid
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNCMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 15 - DNA, 2085 bp, Artificial Sequence
  CPC-P501S polynucleotide
atggccgccgcctacgtgcatagcgacgggagctaccccaaggacaagttcgagaagatcaacgggacatggtactacttcgactcctccggctacatgctcgccgaccgctggcggaagcacaccgacggcaactggtactggttcgataactcgggagagatggccaccggctggaagaagatcgcggacaagtggtactatttcaacgaggagggcgccatgaagaccggctgggtgaagtataaggacacctggtactacctcgacgccaaggagggcgccatgcagtatatcaaggccaacagcaagttcatcggcatcaccgagggagtgatggtcagcaacgcctttatccagagcgccgacggcaccggatggtactacttgaagccggacggcaccctcgcggatcggcccgagatggtgcagcggctgtgggtgtcccggctgctgcgccatagaaaggcccagttgctgctggtgaacctgctgactttcggactggaggtgtgcctggctgccgggatcacgtacgtgccccccctgctgctggaggtgggcgtggaggagaagttcatgacaatggtgctgggcatcggccccgtcctgggcctcgtgtgtgtgcccctcctcgggagtgcgtccgatcattggcggggccgctacggccgccgcagaccgttcatctgggccctgagcctgggcatcctgctctctctcttcctgatcccccgggccggctggctggccggcctgctgtgtcccgacccccgccctctggagctggccctcctgatcctgggcgtgggcctgctggacttctgcggccaggtgtgtttcactcccctggaggctctgctctccgacctcttccgcgaccccgaccactgtaggcaggcttacagcgtgtacgccttcatgatcagtctggggggatgcctgggctatctgctgcccgctatcgactgggacaccagcgccctggccccctacctggggactcaggaggagtgcctgttcggcctgctcaccttgatcttcctgacgtgcgtcgccgccaccctgctggtggccgaggaggcggccctggggcccaccgagcccgccgagggcctgagcgctcccagcctgagcccccattgctgcccgtgcagggctaggctcgccttcaggaatctgggcgctttgctgccccgcctgcatcagctgtgctgtcgcatgcctcgcaccctgcgccgcctgttcgtcgctgagctctgttcctggatggccctgatgacgttcaccctcttctacaccgacttcgtgggggagggcctgtaccagggcgtgcccagggccgagcccggcaccgaggctaggcgccattacgacgagggcgtcaggatgggctctctgggcctcttcctgcagtgcgccatcagtctggtgttctctctggtgatggaccggctggtgcagcgcttcggcacccgggccgtgtacctcgcctctgtggcggctttccccgtcgccgccggcgcgacctgcctgtctcattctgtcgccgtggtgaccgccagcgccgccctgaccggcttcaccttcagtgcgctccagattctgccctacaccctggcgtctctgtaccatcgcgagaagcaggtgttcctgcccaagtaccgcggggacacagggggagcttcctctgaggacagcctgatgaccagcttcttgcccggccccaagccgggggcccctttccccaacggccatgtcggggcgggcggcagcggcctgctccctcccccccccgccctgtgcggcgctagtgcctgcgacgtgagcgtgcgggtggtggtgggggagcccaccgaggctagggtcgtgcctggccgggggatctgcctggacctggccatcctcgactccgccttcctgctctcccaggtggcgcccagcctgttcatgggcagtatcgtgcagctgagccagagcgtgaccgcctacatggtgagcgccgccggcctggggttggtggccatctactttgccacccaggtcgtgttcgacaagagcgatctcgccaagtatagcgcctga

SEQ ID NO: 16 - protein, 694 aa, Artificial Sequence [sequence omitted]

(19) United States
(12) Patent Application Publication
(10) Pub. No.: US 2009/0104153 A1
(43) Pub. Date: Apr. 23, 2009

(54) METHOD OF ELICITING IMMUNE RESPONSE

(76) Inventors: Karen A. Barber, Hertfordshire (GB); Gillian Margaret Baxter, Hertfordshire (GB); Sara Jane Brett, Hertfordshire (GB); Paul Andrew Hamblin, Hertfordshire (GB); John Philip Tite, Hertfordshire (GB); Olivier Vidalin, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/158,427

(22) PCT Filed: Dec. 20, 2006
(86) PCT No.: PCT/GB2006/004818

§ 371 Date: Jun. 20, 2008

Related U.S. Application Data
(60) Provisional application No. 60/752,342, filed on Dec. 21, 2005.

(51) Int. Cl. A61K 31/711; A61K 35/76; A61P 31/18

(52) U.S. Cl. 424/93.2; 514/44

(57) ABSTRACT

The present invention relates to methods of eliciting an immune response by use of a prime-boost schedule for delivering a polynucleotide encoding a heterologous non-self antigen. In particular, the invention relates to a prime-boost schedule wherein the priming polynucleotide composition is delivered by an adenoviral vector, and the boosting polynucleotide composition is coated on or incorporated in a particle and is administered by a particle acceleration device.

The present invention relates to methods of eliciting an immune response by use of a prime-boost schedule for delivering a polynucleotide encoding a heterologous non-self antigen. In particular, the invention relates to a prime-boost schedule wherein the priming polynucleotide composition is delivered by an adenoviral vector, and the boosting polynucleotide composition is coated on or incorporated in a particle and is administered by a particle acceleration device.

Vaccination methods are described in the art, for example see Prayaga et al ((1997) Vaccine 15 (12-13): 1349-1352), Kilpatrick et al (1997) Hybridoma-16: 381-389, Kilpatrick et al (1998) Hybridoma 17: 569-576, Pertmer et al (1995) Vaccine 13; 1427-1430 and Olsen et al (1997) Vaccine 15; 1149-1156. However, there remains a need for optimisation of nucleic acid administration schedules.

Adenoviruses (herein referred to as “Ad” or “Adv”) have a characteristic morphology with an icosohedral capsid consisting of three major proteins, hexon (II), penton base (III) and a knobbed fibre (IV), along with a number of other minor proteins, VI, VIII, IX, IIIa and IVa2 (Russell W. C. 2000, Gen Viriol, 81:2573-2604). The virus genome is a linear, double-stranded DNA with a terminal protein attached covalently to the 5′ termini, which have inverted terminal repeats (ITRs). The virus DNA is intimately associated with the highly basic protein VII and a small peptide termed mu. Another protein, V, is packaged with this DNA-protein complex and provides a structural link to the capsid via protein VI. The virus also contains a virus-encoded protease, which is necessary for processing of some of the structural proteins to produce mature infectious virus.

Over 100 distinct serotypes of adenovirus have been isolated which infect various mammalian species, 51 of which are of human origin. Examples of such adenoviruses from human origin are Ad1, Ad2, Ad4, Ad5, Ad6, Ad11, Ad 24, Ad34, Ad35. The human serotypes have been catagorised into six subgenera (A-F) based on a number of biological, chemical, immunological and structural criteria.

Although Ad5-based vectors have been used extensively in a number of gene therapy trials, there may be limitations on the use of Ad5 and other group C adenoviral vectors due to preexisting immunity in the general population due to natural infection. Ad5 and other group C members tend to be among the most seroprevalent serotypes. Immunity to existing vectors may develop as a result of exposure to the vector during treatment. These types of preexisting or developed immunity to seroprevalent vectors may limit the effectiveness of gene therapy or vaccination efforts. Alternative adenovirus serotypes, thus constitute very important targets in the pursuit of gene delivery systems capable of evading the host immune response.

One such area of alternative serotypes are those of non human primates, especially chimpanzee adenoviruses. See U.S. Pat. No. 6,083,716 which describes the genome of two chimpanzee adenoviruses.

It has been shown that chimpanzee (“Pan” or “C”) adenoviral vectors induce strong immune responses to transgene products as efficiently as human adenoviral vectors (Fitzgerald et al. J. Immunol. 170:1416).

Non human primate adenoviruses can be isolated from the mesenteric lymph nodes of chimpanzees. Chimpanzee adenoviruses are sufficiently similar to human adenovirus subtype C to allow replication of E1 deleted virus in HEK 293 cells. Yet chimpanzee adenoviruses are phylogenetically distinct from the more common human serotypes (Ad2 and Ad5). Pan 6 is less closely related to and is serologically distinct from Pan's 5, 7 and 9.

There are certain size restrictions associated with inserting heterologous DNA into adenoviruses. Human adenoviruses have the ability to package up to 105% of the wild type genome length (Bett et al 1993, J Virol 67 (10), 5911-21). The lower packaging limit for human adenoviruses has been shown to be 75% of the wild type genome length (Parks et al 1995, J Virol 71(4), 3293-8).

Such adenovirus vectors may be formulated with pharmaceutically acceptable excipient, carriers, diluents or adjuvants to produce immunogenic compositions including pharmaceutical or vaccine compositions suitable for the treatment and/or prophylaxis of HIV infection and AIDS.

One example of adenoviruses those which are distinct from prevalent naturally occurring serotypes in the human population such as Ad2 and Ad5. This avoids the induction of potent immune responses against the vector which limits the efficacy of subsequent administrations of the same serotype by blocking vector uptake through neutralizing antibody and influencing toxicity.

Thus, the adenovirus may be an adenovirus which is not a prevalent naturally occurring human virus serotype. Adenoviruses isolated from animals have immunologically distinct capsid, hexon, penton and fibre components but are phylogenetically closely related. Specifically, the virus may be a non-human adenovirus, such as a simian adenovirus and in particular a chimpanzee adenovirus such as Pan 5, 6, 7 or 9. Examples of such strains are described in WO03/000283 and are available from the American Type Culture Collection, University Boulevard, Manassas, Va. 20110-2209, and other sources. Desirable chimpanzee adenovirus strains are Pan 5 [ATCC VR-591], Pan 6 [ATCC VR-592], and Pan 7 [ATCC VR-593].

Chimpanzee adenoviruses are thought to be advantageous over human adenovirus serotypes because of the lack of pre-existing immunity, in particular the lack of cross-neutralising antibodies, to adenoviruses in the target population. Cross-reaction of the chimpanzee adenoviruses with pre-existing neutralizing antibody responses is only present in 2% of the target population compared with 35% in the case of certain candidate human adenovirus vectors. The chimpanzee adenoviruses are distinct from the more common human subtypes Ad2 and Ad5, but are more closely related to human Ad4 of subgroup E, which is not a prevalent subtype. Pan 6 is less closely related to Pan 5, 7 and 9.

Numerous methods of carrying out a particle acceleration approach are known. See for example WO 91/07487. In one illustrative example, gas-driven particle acceleration can be achieved with devices such as those manufactured by Powderject (Chiron Corporation) some examples of which are described in U.S. Pat. Nos. 5,846,796; 6,010,478; 5,865,796; 5,584,807; and EP Patent No. 0500 799. This offers a needle-free delivery approach wherein a dry powder formulation of microscopic particles, coated with a substance such as polynucleotide, is accelerated to high speed within a helium gas jet generated by a hand held device, propelling the particles into a target tissue of interest, typically the skin. In particular, into the epidermis. The particles can be gold beads of about 0.4 to about 4.0 μm diameter, for example 0.6-2.0 μm diameter and the polynucleotide, for example, DNA is coated onto these and then encased in a cartridge for placing into the “gene gun”.

WO9810750 further describes a method for delivering solid particles comprised of nucleic acid molecules to mammalian tissue for the genetic transformation of cells in the tissue with the delivered nucleic acids. In a substantial departure from conventional particle bombardment techniques, the nucleic acid particles transferred using this method are not delivered using dense metal carriers. Furthermore, the molecules have a particle size that is equal to or larger than the average mammalian cell size.

Densified particles comprised of selected nucleic acid molecules and, optionally, suitable carriers or excipients, can be prepared for delivery to mammalian tissue via a needleless syringe which is capable of expelling the particles at supersonic delivery velocities of between Mach 1 and Mach 8. The particles have an average size that is at least about 10 μm, wherein an optimal particle size is usually at least about 10 μm to about 15 μm (equal to or larger than the size of a typical mammalian cell). However, nucleic acid particles having average particle sizes of 250 μm or greater can also be delivered using such methods.

The depth that the delivered particles will penetrate the targeted tissue depends upon particle size (e.g., the nominal particle diameter assuming a roughly spherical particle geometry), particle density, the initial velocity at which the particle impacts the tissue surface, and the density and kinematic viscosity of the tissue. In this regard, optimal individual particle densities (e.g., in contrast to bulk powder density) for use in needleless injection generally range between about 0.1 and 25 g/cm, and injection velocities generally range between about 200 and 3,000 m/sec.

This method can provide targeted delivery of the nucleic acid particles, such as delivery to the epidermis (for example for gene therapy applications) or to the stratum basal layer of skin (for example for nucleic acid immunization applications). Particle characteristics and/or device operating parameters can be selected to provide tissue specific delivery. One particular approach entails the selection of particle size, particle density and initial velocity to provide a momentum density (e.g., particle momentum divided by particle frontal area) of between about 2 and 10 kg/sec/m, and for example between about 4 and about 7 kg/sec/m. Such control over momentum density allows for precisely controlled, tissue-selective delivery of the nucleic acid particles.

The effects of a prime-boost vaccine regimen using DNA delivered by intramuscular injection and recombinant adenovirus in various orders (DNA/Adv, Adv/DNA, DNA/DNA, Adv/Adv and single controls) on the induction of CD4⁺ T-cell responses in the HCV model antigen is described in Park et al (Vaccine 21:4555-4564). The heterologous DNA prime and Adv boost was concluded to be a promising strategy for vaccination regimens for an HCV vaccine.

A mouse malaria model is described in Gilbert et al (Vaccine 20:1039-1045), in which the protective efficacy of DNA delivered by intramuscular injection and recombinant adenovirus and modified vaccinia virus (MVA) by different vaccination regimens (DNA/DNA, Adv/Adv, MVA/MVA, DNA/MVA, DNA/Adv, Adv/DNA, MVA/Adv, Adv/MVA) were examined, and recombinant replication-defective adenoviruses were identified as being useful as boosting agents for strong protective CD8+ T cell responses.

Surprisingly we have found that the combination of the use of an adenoviral vector comprising a polynucleotide encoding a first antigen as a priming composition and the use of particle acceleration techniques to administer a polynucleotide boost gives improved immune responses over single administrations, or alternative prime-boost regimens.

The present invention provides a method of eliciting an immune response in a mammalian subject by administration of an adenoviral vector comprising a polynucleotide encoding a heterologous first non-self antigen, and a subsequent administration of a polynucleotide encoding a heterologous second non-self antigen comprising at least one epitope of the first heterologous non-self antigen, characterised in that the polynucleotide encoding the second heterologous non-self antigen is coated on or incorporated in a particle, and the particle is administered to the subject by a particle acceleration device. In one embodiment of the present invention the particle acceleration device suitable for administering the particle to the subject is a gas-driven device.

As used herein the term “non-self antigen” means an antigen which is not normally present in the mammal to which it is intended to be delivered.

As used herein the term “PMED” means particle-mediated epidermal delivery.

In one embodiment of the present invention, the mammalian subject is human. In a further embodiment, the one or more of the antigens are non-human antigens, i.e. antigens which are not normally expressed in humans. In yet a further embodiment, both the first and second antigens are non-human antigens.

The priming and boosting compositions of the present invention may comprise the same antigens or different forms of the same antigens. The priming composition and the boosting composition will have at least one epitope in common, although it is not necessarily an identical form of the antigen, it may be a different form of the same antigen. An example of different forms of the same antigen is in the case of a polynucleotide encoding a gp120 which lacks a functional signal sequence and is substantially non-glycosylated in mammalian cells, and a polypeptide which is gp120 with its signal sequence and which is glycosylated. A full length and a truncated version of the same protein, or a mutated and a non-mutated form of the same protein, may also be considered different forms of the same antigen for the purposes of a prime-boost format according to the invention.

In one embodiment of the present invention the method is for eliciting a therapeutically effective immune response. In another embodiment the method is for eliciting a protective immune response.

In one embodiment of the present invention the “prime” is a composition comprising a recombinant adenoviral vector comprising a polynucleotide sequence encoding an non-self antigen, which may be incorporated into a plasmid vector, while the “boost” is via particle mediated DNA delivery of a composition comprising the same polynucleotide sequence or a polynucleotide encoding at least one of the same epitopes encoded by the priming composition, for example this epitope may be a T-cell epitope.

In an alternative embodiment of the present invention the polynucleotide boost component is formulated in a particulate formulation suitable for delivery to the epidermis. In one embodiment this is delivered by particle acceleration techniques, for example gas-driven particle acceleration techniques.

In a further embodiment of the invention, one or more adjuvants or polynucleotides encoding an adjuvant may be co-administered with one or more of the prime or boost administrations. Examples of suitable adjuvants include GM-CSF and TLR agonists such as imiquimod. Imiquimod is commercially available as Aldara™ cream (3M). These adjuvants and the combination of these two adjuvant components are described in WO2005025614. In one embodiment one or more adjuvants are co-administered with one or more of the PMED boost administrations only.

One example of adenoviral vectors for use in the invention are non-human primate adenoviruses such as simian adenoviruses, for example chimpanzee adenoviruses as described herein, for example Pan 5, 6, 7 or 9.

The adenovirus of the invention may be replication defective. This means that it has a reduced ability to replicate in non-complementing cells, compared to the wild type virus. This may be brought about by mutating the virus e.g. by deleting a gene involved in replication, for example deletion of the E1a, E1b, E3 or E4 gene.

The adenovirus vectors in accordance with the present invention may be replication defective adenovirus comprising a functional E1 deletion. Thus the adenovirus vectors according to the invention may be replication defective due to the absence of the ability to express adenoviral E1a and E1b, i.e., are functionally deleted in E1a and E1b. The recombinant adenoviruses may also bear functional deletions in other genes [see WO 03/000283] for example, deletions in E3 or E4 genes. The adenovirus delayed early gene E3 may be eliminated from the simian adenovirus sequence which forms part of the recombinant virus. The function of E3 is not necessary to the production of the recombinant adenovirus particle. Thus, it is unnecessary to replace the function of this gene product in order to package a recombinant simian adenovirus useful in the invention. In one particular embodiment the recombinant (simian) adenoviruses have functionally deleted E1 and E3 genes. The construction of such vectors is described in Roy et al., Human Gene Therapy 15:519-530, 2004.

Other recombinant adenoviruses which may be of use in the present invention include those having a functional deletion of the E4 gene, for example a complete functional deletion of E4, or a partial deletion, for example where the E4 ORF6 function is retained. Adenoviral vectors according to the invention may also contain a deletion in the delayed early gene E2a. Deletions may also be made in any of the late genes L1 through to L5 of the simian adenovirus genome. Similarly deletions in the intermediate genes IX and IVa may be useful.

Other deletions may be made in the other structural or non-structural adenovirus genes. The above deletions may be used individually, i.e. an adenovirus sequence for use in the present invention may contain deletions of E1 only. Alternatively, deletions of entire genes or portions thereof effective to destroy their biological activity may be used in any combination. For example in one exemplary vector, the adenovirus sequences may have deletions of the E1 genes and the E4 gene, or of the E1, E2a and E3 genes, or of the E1 and E3 genes (such as functional deletions in E1a and E1b, and a deletion of at least part of E3), or of the E1, E2a and E4 genes, with or without deletion of E3 and so on. Such deletions may be partial or full deletions of these genes and may be used in combination with other mutations, such as temperature sensitive mutations to achieve a desired result.

The adenoviral vectors of the present invention can be produced on any suitable cell line in which the virus is capable of replication. In particular, complementing cell lines which provide the factors missing from the virus vector that result in its impaired replication characteristics can be used. Without limitation, such a cell line may be HeLa [ATCC Accession No. CCL 2], A549 [ATCC Accession No. CCL 185], HEK 293, KB [CCL 17], Detroit [e.g., Detroit 510, CCL 72] and WI-38 [CCL 75] cells, among others. These cell lines are all available from the American Type Culture Collection, 10801 University Boulevard, Manassas, Va. 20110-2209. Other suitable parent cell lines may be obtained from other sources, such as PER.C6© cells, as represented by the cells deposited under ECACC no. 96022940 at the European Collection of Animal Cell Cultures (ECACC) at the Centre for Applied Microbiology and Research (CAMR, UK).

Both the priming composition and the boosting composition may be delivered in more than one dose. Furthermore the initial priming and boosting doses may be followed up with further doses which may be alternated to result in e.g. an Adenovirus prime/PMED DNA boost/further Adenovirus dose/further PMED DNA dose.

There may be additional administrations of DNA between the adenovirus prime and the PMED boost e.g. adenovirus prime followed by DNA delivered intra-dermally, intra-muscularly or sub-cutaneously, followed by PMED DNA boost. There also may be an additional priming dose before the adenovirus prime is administered e.g. DNA delivered intra-dermally, intramuscularly or sub-cutaneously, followed by adenovirus prime then PMED DNA boost.

Examples of treatment regimens of the present invention include

### Adenovirus/PMED

### Adenovirus/PMED/PMED

### Adenovirus/PMED/PMED/PMED

### Adenovirus/Adenovirus/PMED/PMED

### Adenovirus/Adenovirus/PMED/PMED

### Adenovirus/Adenovirus/PMED/PMED/PMED

In one embodiment of the invention one or two priming doses of adenovirus are administered followed by a number of subsequent PMED boosting doses, for example there may be up to 10, or up to 20 or up to 50 PMED boosting doses administered.

The boosting composition should be administered an appropriate time after the priming dose.

An appropriate time is by when the cells have had sufficient time to mature (Wherry, E J Nature Im 2003, 225) from the initial prime vaccination. The amount of time this takes will vary according to many factors, including the nature of the heterologous antigen being delivered, the priming dose, the route by which the prime is administered and the type, age and weight of animal which is being treated. A suitable time may be more than 7 days, 14 days, 21 days, 28 days, 40 days, 100 days, 120 days or more than 180 days, for example between 7 and 40 days, 21 days and 180 days, or between 28 days and 120 days.

Any suitable promoter may be used in the polynucleotides encoding the first or second antigen. One example of a suitable vector is the promoter from the HCMV IE gene, for example wherein the 5′ untranslated region of the HCMV IE gene comprising exon 1 is included and intron A is partially or completely excluded as described in WO 02/36792.

The adenoviral vector can be administered in sufficient amounts to transduce the target cells and to provide sufficient levels of gene transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the retina and other intraocular delivery methods, direct delivery to the liver, inhalation, intranasal, intravenous, intramuscular, intratracheal, subcutaneous, intra-dermal, rectal, oral and other parenteral routes of administration. Routes of administration may be combined, if desired, or adjusted depending upon the gene product or the condition. The route of administration primarily will depend on the nature of the condition being treated.

Dosages of the viral vector will depend primarily on factors such as the condition being treated, the age, weight and health of the patient, and may thus vary among patients. For example, a therapeutically effective adult human or veterinary dosage of the viral vector is generally in the range of from about 100 μL to about 100 mL of a carrier containing concentrations of from about 1×10⁶ to about 1×10¹⁵ particles, about 1×10¹¹ to 1×10¹³ particles, or about 1×10⁹ to 1×10¹² particles virus. Dosages will range depending upon the size of the animal and the route of administration. For example, a suitable human or veterinary dosage (for about an 80 kg animal) for intramuscular injection is in the range of about 1×10⁹ to about 5×10¹² particles per mL, for a single site. Optionally, multiple sites of administration may be delivered. In another example, a suitable human or veterinary dosage may be in the range of about 1×10¹¹ to about 1×10¹⁵ particles for an oral formulation. One of skill in the art may adjust these doses, depending on the route of administration, and the therapeutic or vaccinal application for which the recombinant vector is employed. The levels of expression of the therapeutic product, or for an immunogen, the level of circulating antibody, can be monitored to determine the frequency of dosage administration. Yet other methods for determining the timing of frequency of administration will be readily apparent to one of skill in the art.

As used herein the terms “i.d.” and “ID” represent intra-dermal delivery.

As used herein the terms “i.m.” and “IM” represent intra-muscular delivery.

The methods or compositions of the present invention may be used to protect against or treat disoviral disorders, for example Hepatitis B, Hepatitis C, Human papilloma virus, Human immunodeficiency virus, or Herpes simplex virus; bacterial diseases for example, TB; cancers of the breast, colon, ovary, cervix, and prostate; or autoimmune diseases of asthma, rheumatoid arthritis and Alzheimer's.

It is to be recognised that these specific disease states have been referred to by way of example only, and are not intended to be limiting upon the scope of the present invention.

Suitable heterologous genes to be delivered in such a regimen include those which code for proteins which are capable of eliciting an immune response against a human pathogen, which antigen or antigenic composition is derived from HIV-1, (such as gag, p17, p24, p41, p40, nef, pol, RT, p66, env, gp120 or gp160, gp40, p24, gag, vif, vpr, vpu, rev), human herpes viruses, such as gH, gL gM gB gC gK gE or gD or derivatives thereof or Immediate Early protein such as ICP27, ICP 47, IC P 4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, III and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, eg L1, L2, E1, E2, E3, E4, E5, E6, E7), flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PiIC, adhesins); S. pyogenes (for example M proteins or fragments thereof, C5A protease, S. agalactiae, S. mutans; H. ducreyi; Moraxella spp, including M catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, MPT 44, MPT59, MPT45, HSP10, HSP65, HSP70, HSP 75, HSP90, PPD 19kDa [Rv3763], PPD 38kDa [Rv0934]), M. bovis, M. leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigeila spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; leishmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Specific antigens for M. tuberculosis are for example Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16kDal., Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof where at least two, or for example, three polypeptides of M. tuberculosis are fused into a larger protein. Such fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Examples of suitable antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Examples of bacterial vaccines include antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

The antigens that may be used in the present invention may further comprise antigens derived from parasites that cause Malaria. Antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. Its full structure is disclosed in the International Patent Application No.

PCT/EP92102591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. One embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The invention contemplates the use of an anti-tumour antigen and may be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in Wo99/40188. Other examples of fusion proteins that may contain cancer specific epitopes include bcr/abl fusion proteins.

In one embodiment of the present invention, prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted U.S. Pat. No. 5,955,306), WO 98/20117 (and also the corresponding granted U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (“derivatives”). Such derivatives are suitable for use in therapeutic vaccine formulations which are suitable for the treatment of prostate tumours. Typically the fragment will contain at least 20, for example 50 or 100 contiguous amino acids as disclosed in the above referenced patent and patent applications.

A further example of a prostate antigen is P501S, sequence ID no 113 of WO98/37814, which is incorporated herein by reference. P501S is an example of an antigen for use in the present invention. P501S and constructs thereof are also described in U.S. Pat. No. 6,329,505 also incorporated herein by reference. Immunogenic fragments and portions encoded by the gene thereof comprising at least 20, for example 50 or 100 contiguous amino acids as disclosed in the above referenced patent application, are contemplated. A particular fragment is PS108 (WO 98/50567, incorporated herein by reference).

Other prostate specific antigens are known from WO98/37418, and WO/004149. Another is STEAP PNAS 96 14523 14528 7-12 1999.

Other tumour associated antigens useful in the context of the present invention include: Plu-1 J Biol. Chem 274 (22) 15633-15645, 1999, HASH-1, HasH-2, Cripto (Salomon et al Bioessays 199, 21 61-70,U.S. Pat. No. 5,654,140) Criptin U.S. Pat. No. 5,981,215. Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO/0052165, WO99/33869, WO99/19479, WO 98/45328. Her 2 neu antigens are disclosed inter alia, in U.S. Pat. No. 5,801,005. In one example, the Her 2 neu may comprise the entire extracellular domain (comprising approximately amino acid 1-645) or fragments thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899. One such construct is known as ECD PD, and a second is known as ECD ΔPD. (See WO/00/44899.)

The her 2 neu as used herein can be derived from rat, mouse or human.

The vaccine may also contain antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimer's and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment (AB, the amyloid precursor protein and smaller fragments. This antigen is disclosed in the International Patent Application No. WO99/27944 (Athena Neurosciences).

Potential self-antigens that could be included as vaccines for auto-immune disorders or as a contraceptive vaccine include: cytokines, hormones, growth factors or extracellular proteins, for example a 4-helical cytokine, for example IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The regimens and compositions of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), HIV infections such as AIDS and Hepatitis B (HepB) virus infections.

In one embodiment of the present invention, the heterologous nucleotide sequence encodes one or more of the following antigens:—

HBV—PreS1 PreS2 and Surface env proteins, core and pol

### HCV—Core, E1, E2, P7, NS2, NS3, NS4A, NS4B, NS5A and B

HIV—gp120 gp40, gp160, p17, p24, p41, gag, pol, RT, p66, env, vif, vpr, vpu, tat, rev, nef

### Papilloma—E1, E2, E3, E4, E5, E6, E7, E8, L1, L2

### HSV—gL, gH, gM, gB, gC, gK, gE, gD, ICP47, ICP36, ICP4

Influenza—haemaggluttin, nucleoprotein
TB—Mycobacterial super oxide dismutase, 85A, 85B, MPT44, MPT59, MPT45, HSP10, HSP65, HSP70, HSP90, PPD 19kDa Ag, PPD 38kDa Ag.

Further description of suitable cancer antigens can be found in WO05/025614.

Further description of such suitable HIV antigens can be found in WO03025003.

### HPV Antigens

Antigens of use in the present invention may, for example, be derived from the Human Papilloma Virus (HPV) considered to be responsible for genital warts (HPV 6 or HPV 11 and others), and/or the HPV viruses responsible for cervical cancer (HPV16, HPV18, HPV33, HPV51, HPV56, HPV31, HPV45, HPV58, HPV52 and others). In one embodiment the forms of genital wart prophylactic, or therapeutic, compositions comprise L1 particles or capsomers, and fusion proteins comprising one or more antigens selected from the HPV proteins E1, E2, E5 E6, E7, L1, and L2. In one embodiment the forms of fusion protein are: L2E7 as disclosed in WO 96/26277, and proteinD(1/3)-E7 disclosed in PCT/EP98/05285.

In one embodiment the antigen relevant for HPV cervical infection or cancer, prophylaxis or therapeutic composition may comprise HPV 16 or 18 antigens. For example, L1 or L2 antigen monomers, or L1 or L2 antigens presented together as a virus like particle (VLP) or the L1 alone protein presented alone in a VLP or capsomer structure. Such antigens, virus like particles and capsomer are per se known. See for example WO94/00152, WO94/20137, WO94/05792, and WO93/02184. Additional early proteins may be included alone or as fusion proteins such as E7, E2 or for example E5; One embodiment of this includes a VLP comprising L1E7 fusion proteins (WO 96/11272). In one embodiment the HPV 16 antigens comprise the early proteins E6 or E7 in fusion with a protein D carrier to form Protein D-E6 or E7 fusions from HPV 16, or combinations thereof; or combinations of E6 or E7 with L2 (WO 96/26277). Alternatively the HPV 16 or 18 early proteins E6 and E7, may be presented in a single molecule, for example as a Protein D-E6/E7 fusion. Such a composition may optionally provide either or both E6 and E7 proteins from HPV 18, for example in the form of a Protein D-E6 or Protein D-E7 fusion protein or Protein D E6/E7 fusion protein.

### HIV Antigens

In a particular suitable embodiment of the invention, the first and second polypeptide antigens are selected from HIV derived antigens, particularly HIV-1 derived antigens. HIV Tat and Nef proteins are early proteins, that is, they are expressed early in infection and in the absence of structural protein.

The Nef gene encodes an early accessory HIV protein which has been shown to possess several activities. For example, the Nef protein is known to cause the removal of CD4, the HIV receptor, from the cell surface, although the biological importance of this function is debated. Additionally Nef interacts with the signal pathway of T cells and induces an active state, which in turn may promote more efficient gene expression. Some HIV isolates have mutations in this region, which cause them not to encode functional protein and are severely compromised in their replication and pathogenesis in vivo.

The Gag gene is translated from the full-length RNA to yield a precursor polyprotein which is subsequently cleaved into 3-5 capsid proteins; the matrix protein, capsid protein and nucleic acid binding protein and protease. (Fundamental Virology, Fields B N, Knipe D M and Howley M 1996 2. Fields Virology vol 2 1996).

The Gag gene gives rise to the 55-kilodalton (kD) Gag precursor protein, also called p55, which is expressed from the unspliced viral mRNA. During translation, the N terminus of p55 is myristoylated, triggering its association with the cytoplasmic aspect of cell membranes. The membrane-associated Gag polyprotein recruits two copies of the viral genomic RNA along with other viral and cellular proteins that triggers the budding of the viral particle from the surface of an infected cell. After budding, p55 is cleaved by the virally encoded protease (a product of the Pol gene) during the process of viral maturation into four smaller proteins designated MA (matrix [p17]), CA (capsid [p24]), NC (nucleocapsid [p9]), and p6.(4).

In addition to the 3 major Gag proteins (p17, p24 and p9), all Gag precursors contain several other regions, which are cleaved out and remain in the virion as peptides of various sizes. These proteins have different roles e.g. the p2 protein has a proposed role in regulating activity of the protease and contributes to the correct timing of proteolytic processing.

The MA polypeptide is derived from the N-terminal, myristoylated end of p55. Most MA molecules remain attached to the inner surface of the virion lipid bilayer, stabilizing the particle. A subset of MA is recruited inside the deeper layers of the virion where it becomes part of the complex which escorts the viral DNA to the nucleus. These MA molecules facilitate the nuclear transport of the viral genome because a karyophilic signal on MA is recognized by the cellular nuclear import machinery. This phenomenon allows HIV to infect non-dividing cells, an unusual property for a retrovirus.

The p24 (CA) protein forms the conical core of viral particles. Cyclophilin A has been demonstrated to interact with the p24 region of p55 leading to its incorporation into HIV particles. The interaction between Gag and cyclophilin A is essential because the disruption of this interaction by cyclosporin A inhibits viral replication.

The NC region of Gag is responsible for specifically recognizing the so-called packaging signal of HIV. The packaging signal consists of four stem loop structures located near the 5′ end of the viral RNA, and is sufficient to mediate the incorporation of a heterologous RNA into HIV-1 virions. NC binds to the packaging signal through interactions mediated by two zinc-finger motifs. NC also facilitates reverse transcription.

The p6 polypeptide region mediates interactions between p55 Gag and the accessory protein Vpr, leading to the incorporation of Vpr into assembling virions. The p6 region also contains a so-called late domain which is required for the efficient release of budding virions from an infected cell.

The Pol gene encodes three proteins having the activities needed by the virus in early infection, reverse transcriptase RT, protease, and the integrase protein needed for integration of viral DNA into cellular DNA. The primary product of Pol is cleaved by the virion protease to yield the amino terminal RT peptide which contains activities necessary for DNA synthesis (RNA and DNA directed DNA polymerase, ribonuclease H) and carboxy terminal integrase protein. HIV RT is a heterodimer of full-length RT (p66) and a cleavage product (p51) lacking the carboxy terminal Rnase integrase domain.

RT is one of the most highly conserved proteins encoded by the retroviral genome. Two major activities of RT are the DNA Pol and Ribonuclease H. The DNA Pol activity of RT uses RNA and DNA as templates interchangeably and like all DNA polymerases known is unable to initiate DNA synthesis de novo, but requires a pre existing molecule to serve as a primer (RNA).

The Rnase H activity inherent in all RT proteins plays the essential role early in replication of removing the RNA genome as DNA synthesis proceeds. It selectively degrades the RNA from all RNA-DNA hybrid molecules. Structurally the polymerase and ribo H occupy separate, non-overlapping domains within the Pol covering the amino two thirds of the Pol. The p66 catalytic subunit is folded into 5 distinct subdomains. The amino terminal 23 of these have the portion with RT activity. Carboxy terminal to these is the Rnase H Domain. After infection of the host cell, the retroviral RNA genome is copied into linear double stranded DNA by the reverse transcriptase that is present in the infecting particle. The integrase (reviewed in Skalka A M '99 Adv in Virus Res 52 271-273) recognises the ends of the viral DNA, trims them and accompanies the viral DNA to a host chromosomal site to catalyse integration. Many sites in the host DNA can be targets for integration. Although the integrase is sufficient to catalyse integration in vitro, it is not the only protein associated with the viral DNA in vivo—the large protein-viral DNA complex isolated from the infected cells has been denoted the pre integration complex. This facilitates the acquisition of the host cell genes by progeny viral genomes.

The integrase is made up of 3 distinct domains, the N terminal domain, the catalytic core and the C terminal domain. The catalytic core domain contains all of the requirements for the chemistry of polynucleotidyl transfer.

HIV-1 derived antigens for us in the invention may thus for example be selected from Gag, Tat, p17 (a portion of Gag), p24 (another portion of Gag), p41, p40, Nef, Pol, RT (a portion of Pol), p66 (a portion of RT), Env, gp120, gp140 or gp160, gp40, p24, vif, vpr, vpu, rev and immunogenic derivatives thereof and immunogenic fragments thereof, particularly Gag, Nef and Pol and immunogenic derivatives thereof and immunogenic fragments thereof including p17, p24 and RT. HIV vaccines may comprise polypeptides and/or polynucleotides encoding polypeptides corresponding to multiple different HIV antigens for example 2 or 3 or 4 or more HIV antigens which may be selected from the above list. Several different antigens may, for example, be comprised in a single fusion protein.

For example an antigen may comprise Gag or an immunogenic derivative or immunogenic fragment thereof, fused to RT or an immunogenic derivative or immunogenic fragment thereof, fused to Nef or an immunogenic derivative or immunogenic fragment thereof wherein the Gag portion of the fusion protein is present at the 5′ terminus end of the polypeptide.

A Gag sequence of use according to the invention may exclude the Gag p6 polypeptide encoding sequence. A particular example of a Gag sequence for use in the invention comprises p17 and/or p24 encoding sequences.

A RT sequence may contain a mutation to substantially inactivate any reverse transcriptase activity. One particular inactivation mutation involves the substitution of W tryptophan 229 for K (lysine), see WO03/025003.

An RT sequence may also or instead contain a mutation at position 592 corresponding to Met eg a mutation to Lys. The purpose of this mutation is to remove a site which serves as an internal initiation site in prokaryotic expression systems.

The RT gene is a component of the bigger Pol gene in the HIV genome. It will be understood that the RT sequence employed according to the invention may be present in the context of Pol, or a fragment of Pol corresponding at least to RT. Such fragments of Pol retain major CTL epitopes of Pol. In one specific example, RT is included as just the p51 or just the p66 fragment of RT.

The RT component of the fusion protein or composition according to the invention optionally comprises a mutation at position 592, or equivalent mutation in strains other than HXB2, such that the methionine is removed by mutation to another residue e.g. lysine. The purpose of this mutation is to remove a site which serves as an internal initiation site in prokaryotic expression systems.

Optionally the Nef sequence for use in the invention is truncated to remove the sequence encoding the N terminal region i.e. removal of from 30 to 85 amino acids, for example from 60 to 85 amino acids, particularly the N terminal 65 amino acids (the latter truncation is referred to herein as trNef). Alternatively or additionally the Nef may be, modified to remove one or more myristylation sites. For example the Gly 2 myristylation site may be removed by deletion or substitution. Alternatively or additionally the Nef may be modified to alter the dileucine motif of Leu 174 and Leu 175 by deletion or substitution of one or both leucines. The importance of the dileucine motif in CD4 downregulation is described e.g. in Bresnahan P. A. et al (1998) Current Biology, 8(22): 1235-8.

Antigens according to the invention may comprise Gag, Pol and Nef wherein at least 75%, or at least 90% or at least 95%, for example, 96% of the CTL epitopes of these native antigens are present.

Antigens of use in the present invention may comprise p17/p24 Gag, p66 RT, and truncated Nef as defined above, 96% of the CTL epitopes of the native Gag, Pol and Nef antigens may be present.

Specific polynucleotide constructs and corresponding polypeptide antigens which can be used according to the invention include:

1. p17, p24 (codon optimised) Gag-p66 RT (codon optimised)-truncatedNef;
2. truncatedNef-p66 RT (codon optimised)-p17, p24 (codon optimised) Gag;
3. truncatedNef-p17, p24 (codon optimised) Gag-p66 RT (codon optimised);
4. p66 RT (codon optimised) p17, p24 (codon optimised) Gag-truncatedNef;
5. p66 RT (codon optimised)-truncatedNef-p17, p24 (codon optimised) Gag;
6. p17, p24 (codon optimised) Gag-truncatedNef-p66 RT (codon optimised).

It is envisaged that the present invention may be effective at breaking tolerance against self-antigens, for example the cancer antigens P501S, or MUC-1.

The heterologous genes encoding such antigens, may encode immunogenic derivatives or immunogenic fragments thereof rather than the whole antigen.

It will be understood that for all of the heterologous sequences included in the invention, these do not necessarily represent sequences encoding the full length or native proteins. Immunogenic derivatives such as truncated or otherwise altered e.g. mutated proteins are also contemplated, as are fragments which encode at least one epitope, for example a CTL epitope, typically a peptide of at least 8 amino acids. Polynucleotides which encode a fragment of at least 8, for example 8-10 amino acids or up to 20, 50, 60, 70, 100, 150 or 200 amino acids in length are considered to fall within the scope of the invention as long as the encoded oligo or polypeptide demonstrates antigenicity, that is to say that the major CTL epitopes are retained by the oligo or polypeptide.

The polypeptide molecules encoded by the polynucleotide sequences according to the invention may represent a fragment of for example 50% of the length of the native protein, which fragment may contain mutations but which retains at least one epitope and demonstrates antigenicity. Similarly, immunogenic derivatives according to the invention must demonstrate antigenicity. Immunogenic derivatives may provide some potential advantage over the native protein such as reduction or removal of a function of the native protein which is undesirable in a vaccine antigen such as enzyme activity (for example, RT), or CD4 downregulation (for example, Nef).

The polynucleotide sequences may be codon optimised for mammalian cells. Such codon-optimisation is described in detail in WO05/025614.

In one embodiment of the present invention the constructs comprise an N-terminal leader sequence. The signal sequence, transmembrane domain and cytoplasmic domain are individually all optionally present or deleted. In one embodiment of the present invention all these regions are present but modified.

The present invention comprises the method of treatment of a mammalian subject for treating HIV by administration of a first composition comprising an adenoviral vector comprising a polynucleotide encoding a heterologous non-self antigen capable of raising an immune response and subsequently, a second composition comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device.

The present invention further comprises a kit comprising a first composition comprising an adenoviral vector comprising a polynucleotide encoding a heterologous non-self antigen capable of raising an immune response, and a second composition comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device for use in medicine.

The present invention further comprises a kit comprising a first vaccine comprising an adenoviral vector comprising a polynucleotide encoding a heterologous non-self antigen capable of raising an immune response and a second vaccine comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device.

### EXAMPLES

### Example 1

### Construction of Ovalbumin Expressing Vectors

A gene encoding a non-secreted form of chicken ovalbumin was constructed by deleting the secretion signal (a.a. 20-145) of the wild type chicken ova gene. This truncated gene is termed OVAcyt to signify that it is a non-secreted, cytoplasmic form of the ovalbumin protein. This gene (see FIG. 1) was amplified by PCR using primers incorporating restriction sites to enable ligation into the DNA vaccine vector p7313 (Full details of this plasmid are given in WO2004041852)

A gene encoding a secreted form of chicken ovalbumin was constructed by inserting the ovalbumin gene (see FIG. 2) into a modified eukaryotic expression vector optimised for DNA vaccination based on pCI plasmid (promega).

### Example 2

### Construction of Gag, RT, Nef Plasmid

Plasmid p73i-Tgrn
1. Plasmid: p73i-GRN2 Clone #19 (p17/p24(opt)/RT(opt)trNef)-Repaired

### Gene of Interest:

The p17/p24 portion of the codon optimised Gag, codon optimised RT and truncated Nef gene from the HIV-1 clade B strain HXB2 downstream of an Iowa length HCMV promoter+exon1, and upstream of a rabbit β-globin poly-adenylation signal.

Plasmids containing the trNef gene derived from plasmid p17/24trNef1 contain a PCR error that gives an R to H amino acid change 19 amino acids from the end of Nef. This was corrected by PCR mutagenesis, the corrected Nef PCR stitched to codon optimised RT from p7077-RT3, and the stitched fragment cut with ApaI and BamHI, and cloned into ApaI/BamHI cut p73i-GRN.

### Primers:

PCR coRT from p7077-RT3 using primers:
(Polymerase=PWO (Roche) throughout.

Sense: U1
GAATTCGCGGCCGCGATGGGCCCCATCAGTCCCATCGAGACCGTGCCGGT
GAAGCTGAAACCCGGGAT
AScoRT-Nef
GGTGTGACTGGAAAACCCACCATCAGCACCTTTCTAATCCCCGC

Cycle: 95° C. (30 s) then 20 cycles 95° C. (30 s), 55° C. (30 s), 72° C. (180 s), then 72° C. (120 s) and hold at 4° C.

The 1.7 kb PCR product was gel purified.

PCR 5′ Nef from p17/24trNef1 using primers:

Sense: S-Nef
ATGGTGGGTTTTCCAGTCACACC
Antisense: ASNef-G:
GATGAAATGCTAGGCGGCTGTCAAACCTC

Cycle: 95° C. (30 s) then 15 cycles 95° C. (30 s), 55° C. (30 s), 72° C. (60 s), then 72° C. (120 s) and hold at 4° C.
PCR 3′ Nef from p17/24trNef1 using primers:

Sense: SNEF-G
GAGGTTTGACAGCCGCCTAGCATTTCATC
Antisense:
AStrNef (antisense)
CGCGGATCCTCAGCAGTTCTTGAAGTACTCC

Cycle: 95° C. (30 s) then 15 cycles 95° C. (30 s), 55° C. (30 s), 72° C. (60 s), then 72° C. (120 s) and hold at 4° C.

The PCR products were gel purified. Initially the two Nef products were stitched using the 5′ (S-Nef) and 3′ (AstrNef) primers.

Cycle: 95° C. (30 s) then 15 cycles 95° C. (30 s), 55° C. (30 s), 72° C. (60 s), then 72° C. (180 s) and hold at 4° C.

The PCR product was PCR cleaned, and stitched to the RT product using the U1 and AstrNef primers:

Cycle: 95° C. (30 s) then 20 cycles 95° C. (30 s), 55° C. (30 s), 72° C. (180 s), then 720° C. (180 s) and hold at 4° C.

The 2.1 kb product was gel purified, and cut with ApaI and BamHI. The plasmid p73I-GRN was also cut with Apa1 and BamHI gel purified and ligated with the ApaI-Bam RT3trNef to regenerate the p17/p24(opt)/RT(opt)trNef gene.

2. Plasmid: p73I-RT w229k (Inactivated RT)

### Gene of Interest:

Generation of an inactivated RT gene downstream of an Iowa length HCMV promoter+exon 1, and upstream of a rabbit β-globin poly-adenylation signal.

Due to concerns over the use of an active HIV RT species in a therapeutic vaccine inactivation of the gene was desirable. This was achieved by PCR mutagenesis of the RT (derived from P73I-GRN2) amino acid position 229 from Trp to Lys (R7271 p1-28).

### Primers:

PCR 5′ RT+mutation using primers:
(polymerase=PWO (Roche) throughout)

Sense: RT3-u:1
GAATTCGCGGCCGCGATGGGCCCCATCAGTCCCATCGAGACCGTGCCGGT
GAAGCTGAAACCCGGGAT
Antisense: AScoRT-Trp229Lys
GGAGCTCGTAGCCCATCTTCAGGAATGGCGGCTCCTTCT

### Cycle:

1×[94° C. (30 s)]
15×[94° C. (30 s)/55° C. (30 s)/72° C. (60 s)]
1×[72° C. (180 s)]

### PCR Gel Purify

PCR 3′ RT+mutation using primers:

Antiense: RT3-I:1
GAATTCGGATCCTTACAGCACCTTTCTAATCCCCGCACTCACCAGCTTGT
CGACCTGCTCGTTGCCGC
Sense: ScoRT-Trp229Lys
CCTGAAGATGGGCTACGAGCTCCATG

### Cycle:

1×[94° C. (30 s)]
15×[94° C. (30 s)/55° C. (30 s)/72° C. (60 s)]
1×[72° C. (180 s)]

### PCR Gel Purify

The PCR products were gel purified and the 5′ and 3′ ends of RT were stitched using the 5′ (RT3-U1) and 3′ (RT3-L1) primers.

### Cycle:

1×[94° C. (30 s)]
15×[94° C. (30 s)/55° C. (30 s)/72° C. (120 s)]
1×[72° C. (180 s)]

The PCR product was gel purified, and cloned into p7313ie, utilising NotI and BamHI restriction sites, to generate p73I-RT w229k. (See FIG. 3)

3. Plasmid: p73i-Tgrn

### Gene of Interest:

The p17/p24 portion of the codon optimised gag, codon optimised RT and truncated Nef gene from the HIV-1 clade B strain HXB2 downstream of an Iowa length HCMV promoter+exon1, and upstream of a rabbit β-globin poly-adenylation signal.

Triple fusion constructs which contain an active form of RT, may not be acceptable to regulatory authorities for human use thus inactivation of RT was achieved by Insertion of a NheI and ApaI cut fragment from p73i-RT w229k, into NheI/ApaI cut p73i-GRN2#19 (FIG. 4). This results in a W→K change at position 229 in RT.

The full sequence of the Tgrn plasmid insert is shown in FIG. 3. This contains p17 p24 (opt) Gag, p66 RT (opt and inactivated) and truncated Nef.

Alternative constructs of Gag, RT and Nef are as follows:

trNef-p66 RT (opt)-p17, p24 (opt) Gag,
trNef-p17, p24 (opt) Gag-p66 RT (opt),
p66 RT (opt)-p17, p24 (opt) Gag-trNef,
p66 RT (opt)-trNef-p17, p24 (opt) Gag,
p17, p24 (opt) Gag-trNef-p66 RT (opt).

Full sequences for these constructs are given in FIGS. 4 to 8 respectively.

(This is described in full in WO03/025003)

### Example 3

### Construction of GM-CSF Plasmid

Mouse GM-CSF was cloned from a cDNA library and cloned into the expression vector pVACss2. This cDNA clone was used as a template to amplify the mGM-CSF open reading frame by PCR, using primers incorporating a Kozac sequence, start codon and restriction enzyme sites to enable cloning into the DNA vaccine vector p7313

This is described in full in WO02/08435 and WO05/025614.

### Example 4

### Construction of E1/E3 Deleted Adv5-OVA

The replication-deficient partially deleted (E1a⁻, E1b⁻, partial E3⁻) adenovirus serotype 5 vectors Ad-OVA (carrying the cDNA for secreted OVA) were constructed as previously described in Vaccine 21 (2002) 231-242.

### Example 5

### Construction of the E11/E3 Deleted Pan Adenovirus

### Generation of Recombinant E1-Deleted SV-25 Vector:

A plasmid was constructed containing the complete SV-25 genome except for an engineered E1 deletion. At the site of the E1 deletion recognition sites for the restriction enzymes I-CeuI and PI-SceI which would allow insertion of transgene from a shuttle plasmid where the transgene expression cassette is flanked by these two enzyme recognition sites were inserted.

A synthetic linker containing the restriction sites SwaI-SnaBI-SpeI-AflII-EcoRV-SwaI was cloned into pBR322 that was cut with EcoRI and NdeI. This was done by annealing together two synthetic oligomers SV25T (5′-AAT TTA MT ACG TAG CGC ACT AGT CGC GCT AAG CGC GGA TAT CAT TTA AA-3′) and SV25B (5′-TAT TTA AAT GAT ATC CGC GCT TAA GCG CGA CTA GTG CGC TAC GTA TTT A-3′) and inserting it into pBR322 digested with EcoRI and NdeI. The left end (bp1 to 1057) of Ad SV25 was cloned into the above linker between the SnaBI and SpeI sites. The right end (bp28059 to 31042) of Ad SV25 was cloned into the linker between the AflII and EcpRV sites. The adenovirus E1 was then excised between the EcoRI site (bp 547) to XhoI (bp 2031) from the cloned left end as follows. A PCR generated I-CeuI-PI-SceI cassette from pShuttle (Clontech) was inserted between the EcoRI and SpeI sites. The 10154 bp XhoI fragment of Ad SV-25 (bp2031 to 12185) was then inserted into the SpeI site. The resulting plasmid was digested with HindIII and the construct (pSV25) was completed by inserting the 18344 bp Ad SV-25 HindIII fragment (bp11984 to 30328) to generate a complete molecular clone of E1 deleted adenovirus SV25 suitable for the generation of recombinant adenoviruses. Optionally, a desired transgene is inserted into the I-CeuI and PI-SceI sites of the newly created pSV25 vector plasmid.

To generate an AdSV25 carrying a marker gene, a GFP (green fluorescent protein) expression cassette previously cloned in the plasmid pShuttle (Clontech) was excised with the restriction enzymes I-CeuI and PI-SceI and ligated into pSV25 (or another of the Ad chimp plasmids described herein) digested with the same enzymes. The resulting plasmid (pSV25GFP) was digested with SwaI to separate the bacterial plasmid backbone and transfected into the E1 complementing cell line HEK 293. About 10 days later, a cytopathic effect was observed indicating the presence of replicative virus. The successful generation of an Ad SV25 based adenoviral vector expressing GFP was confirmed by applying the supernatant from the transfected culture on to fresh cell cultures. The presence of secondarily infected cells was determined by observation of green fluorescence in a population of the cells.

### Construction of E3 Deleted Pan-6 and Pan-7 Vectors:

In order to enhance the cloning capacity of the adenoviral vectors, the E3 region can be deleted because this region encodes genes that are not required for the propagation of the virus in culture. Towards this end, E3-deleted versions of Pan-5, Pan-6, Pan-7, and C68 have been made (a 3.5 kb Nru-AvrII fragment containing E31-9 is deleted).

### E3 Deletion in Pan6 Based Vector:

E1-deleted pPan6-pkGFP molecular clone was digested with Sbf I and Not I to isolate 19.3 kb fragment and ligated back at Sbf I site. The resulting construct pPan6-Sbf I-E3 was treated with Eco 47 III and Swa I, generating pPan6-E3. Finally, 21 kb Sbf I fragment from Sbf I digestion of pPan6-pkGFP was subcloned into pPan6-E3 to create pPan6-E3-pkGFP with a 4 kb deletion in E3.

### E3 Deleted Pan7 Vector:

The same strategy was used to achieve E3 deletions in Pan 7. First, a 5.8 kb Avr II fragment spanning the E3 region was subcloned pSL-1180, followed by deletion of E3 by Nru I digestion. The resulting plasmids were treated with Spe I and Avr II to obtain 4.4 kb fragments and clone into pPan7-pkGFP at Avr II sites to replace the original E3 containing Avr II fragments, respectively. The final pPan7-E3-pkGFP construct had a 3.5 kb E3-deletion.

A full description of construction of E1, E3 and E4 deletions in these and other Pan Adenovirus serotypes is given in WO03/0046124. Further information is also available in Human Gene Therapy 15:519-530.

### Example 6

### Insertion of Gag, RT, Nef Sequence into Adenovirus

Subcloning of GRN Expression Cassette into pShuttle Plasmid:

The entire expression cassette consisting of promoter, cDNA and polyadenylation signal was isolated from pT-GRN constructs by Sph I and EcoR I double digestion. The Sph I end of the Sph I/EcoR I fragment was filled in with Klenow and cloned into pShuttle plasmid at EcoR I and Mlu I sites where the Mlu I end was blunted.

During the cloning process an additional flanking sequence became associated with the HIV expression cassette. This sequence is known as the Cer sequence and has no known function.

Transfer of GRN Expression Cassette into E1/E3-Deleted Molecular Clones of Pan6 and Pan7 Vectors:

The expression cassette was retrieved from pShuttle by I-Ceu I and PI-Sce I digestions and cloned into the same sites of the molecular clones of Pan6 and Pan7 vectors. Recombinant clones were identified through green/white selection and confirmed by extensive restriction enzyme analysis.

### Rescue and Propagation of Recombinant Viruses:

Molecular clones of C6 and C7 vectors were treated with appropriate restriction endonucleases (PmeI and PacI respectively) to release intact linear vector genomes and transfected into 293 cells using the calcium phosphate method. When full cytopathetic effect was observed in the transfected cells, crude viral lysate was harvested and gradually expanded to large scale infections in 293 cells (1×10e9 cells). Viruses from large scale infections were purified by standard CsCl sedimentation method.

In addition the pShuttle plasmid can be further trimmed by cutting with EcoRI and XmnI to remove a 3′ linker sequence and reduce the plasmid size to produce pShuttleGRNc. This modified plasmid can be used to generate an additional Pan7 virus (C7-GRNc) using the method as described above.

Other constructs were similarly inserted into both the Pan 6 and Pan 7 adenovirus. However Pan 6 with a p66 RT (opt)-trNef-p17, p24 (opt) Gag insert was not successfully produced.

### Example 7

### Mixed Modality Vaccination PMED/Adv5 Evaluation of Immunogenicity

The potential of mixed modality vaccination was evaluated by combining DNA delivery (PMED) and viral vector (Adv5). Animals were primed either with DNA (1 μg) or Adenovirus (5×10⁶ pfu or 1×10⁸ pfu) expressing OVASec. Three weeks later the animals received a boost with homologous regimen (DNA primed boost DNA or Adv primed boost Adv) or heterologous regimen (DNA primed boost Adv or Adv primed boosted DNA). Animals were culled 6 days post-boost and cellular responses were monitored. The cytokine production monitored by intracellular staining for OVA specific CD8 T-cells have shown a clear enhancement of responses (see FIG. 9) when animals are primed with Adv followed by a PMED boost. In the group of mice primed with adenovirus using either 10⁸ pfu or 5×10⁶ pfu followed with a DNA PMED boost there was a higher percentage (range from 2.1% to 2.3%) of CD8 specific T-cell producing IFNγ after stimulation with OVA specific peptide (SIINFEKL). Groups of mice that were immunised with one modality only have shown a more reduced cellular response to the specific peptide stimulation (range from 0.9% to 1.2%). The lowest responses were observed when PMED was used to prime animals followed with an Adenovirus boost.

The kinetics of responses after mixed modality delivery were compared (See FIG. 10). The responses were followed using tetramer staining and different intervals between prime and boosted have been tested. Animals were primed either with PMED DNA (1 μg) or Adv5-OVA vector (5×10⁶ pfu) expressing OVASec and boosted either day 21 and/or day 110. A faster development of immune response was observed against the ovalbumin antigen day 8 post first boost in the group of mice primed with Adv5-OVA followed with DNA PMED boost. In the group of mice primed with DNA followed by Adv, responses were delayed and the level of response was similar to the response induced after a single Adv prime. In both groups of mice immunised with a mixed modality regimen responses were sustained for a long period of time after the boost.

When different intervals were compared for prime and boost, animals primed with Adv followed by DNA boost day 110 mounted very high responses (up to 30.5% OVA specific CD8⁺ cells). These were similar to the responses observed in animals immunised three times

The responses in animals primed with Adv only were measured up to day 70 with a slower slope appearing by day 35.

### Example 8

### Impact of the Route of Delivery in DNA/Adv Mixed Modality Vaccine

Several sequences of immunisation and different route of delivery were compared for DNA using the chicken ovalbumin model antigen. Mice were primed either with adenovirus (Adv5, intramuscular, 5×10⁶ pfu) and boosted (day 35) with DNA delivered by PMED (1 μg), intra-muscular (100 μg DNA in PBS 1×), intra-dermal (100 μg DNA in PBS 2×). These groups were compared to animals primed with DNA (PMED, intramuscular, or intra-dermal) and boosted by Adv5 (intramuscular). Immune responses were monitored using OVA specific tetramers in blood from immunised animals prior and after boost (see FIG. 11).

The primed animals showed a relatively low level of specific tetramers before boost (not more than 5% in Adv immunised groups). In the groups of animals primed with Adv (red bars) there was an increase of the magnitude of responses after PMED boost compare to other DNA modalities (intramuscular and intra-dermal). In the groups of DNA primed animals (blue bars), a boosting effect on responses by adenovirus was seen in all the groups with a higher magnitude of response in animals primed with DNA intramuscularly.

A final DNA boost was given and CD8 and CD4 responses were monitored by intracellular staining (see FIG. 12). Animals primed with Adv and boosted with DNA PMED twice developed very high CD8 responses (up to 35%) as well as CD4 responses (up to 24%) in comparison with all the other groups.

In this experiment we have confirmed previous findings on the potential of Adv primed followed with PMED boost and compared it directly to DNA intramuscular primed animals followed with Adv boost. Our results have reproduced what has been described in the literature with DNA delivered intramuscular and bring a new finding concerning the potential of Adv to enhance immune responses (CD8 and CD4) when its used to prime immune responses prior DNA PMED boost.

### Example 9

### Potential of DNA/Adv Mixed Modality Vaccine and Molecular Adjuvant

The potential of mixed modality Adenovirus/DNA PMED when DNA is adjuvantised was assessed. Mice primed with Adv5-OVA were boosted with DNA (expressing OVA cytoplasmic or secreted form). The DNA was either DNA plasmid encoding for the OVA antigen alone, or co-precipitation of both DNA expressing OVA antigen and GM-CSF. In one group of animals, Aldara™ cream was applied to the immunisation spot 24 hours after PMED delivery on the immunisation spot, the other group of animals were not treated with imiquimod. Cellular responses were monitored by tetramer staining and flow cytometry or IFNγ and IL2 cytokines. Animals which received PMED DNA expressing OVA and GM-CSF followed with Imiquimod treatment have developed higher specific CD8⁺ T-cell responses for Ovalbumin (Tetramers responses shown FIG. 13—top panel). The high frequency of OVA specific cells induced after Adv/DNA regimen were enhanced further when both adjuvants were provided with the DNA boost. The same observation was made by monitoring T-cells producing specifically cytokines in responses to CD4 or CD8 specific OVA peptides (see FIG. 13—bottom panel). Responses for both populations CD8 and CD4 cells were enhanced (from 2 to 3 folds) in comparison with mixed modality regimen without adjuvant when both adjuvants are used with a range from 33 to 41% CD8⁺ specific cells and a range from 29 to 32% CD4⁺ specific cells producing IFNγ. Other groups of mice display a high but more reduced percentage of CD8⁺ and CD4⁺ T-cells producing cytokines.

In this experiment we have demonstrated Adenovirus prime DNA boost mixed modality offer an advantage in enhancement of immune responses over DNA alone or adenovirus alone. These responses can be increased further when DNA is adjuvantised with both adjuvants GM-CSF and Imiquimod.

### Example 10

### Evaluation of NHP Adenovirus

The capabilities of viral NHP vectors in combination with PMED delivery to mount immune responses against HIV antigens were measured and compared with the data generated previously in the Adv5 OVA model.

### Mixed Modality Delivery Using NHP Adenovirus Delivered Intra-Muscularly in Mice

The induction of immune responses after homologous prime/boost NHP delivery and mixed modality Adv/PMED was measured. Different routes of immunisation were used to deliver the Adenovirus (intramuscular and intra-dermal). Mice were immunised with 1×10⁹ particles of NHP-Adv and of 1 μg of DNA PMED expressing HIV antigens and prime and boost immunisations were given with an interval of 42 days. Improved responses were observed against all three antigens (RT, Gag and Nef) when mixed modality (Adv/PMED) was compared to homologous delivery (adv/adv and PMED/PMED). Mixed modality regimen enhanced up to 4 fold CD8 responses and up to 7 fold CD4 responses. Responses against RT antigens are illustrated in FIG. 14. No differences were observed when different routes of delivery were used for NHP vectors.

Mixed modality delivery using NHP adv vectors and DNA delivered intramuscular, intra-dermal or PMED was compared. In this study, groups of mice were primed intramuscular with 1×10⁹ particles of NHP-HIV adenovirus and boosted 42 days later by either PMED DNA (1 μg), intramuscular DNA in saline (100 μg) or intra-dermal DNA in saline 2× (100 μg) expressing HIV antigens.

Animals primed with adenovirus followed with a PMED DNA boost exhibited a higher level of cellular responses. Typical responses observed are illustrated with RT antigen in FIG. 15, CD8 T-cells producing mainly IFNγ (range from 7% to 12% RT specific CD8+ cells producing INFγ) and CD4 T-cells producing both IFNγ and IL2 (range from 0.35 to 2.2% RT specific CD4⁺ cells producing both IL2 and IFNγ). Other groups adenovirus primed animals boosted with DNA in saline solution showed a reduced percentage of CD8 cells producing cytokines (range from 1.5 to 3.5% RT specific CD8⁺ cells producing IFNγ). The mixed modalities for Adv/DNA show the ability to enhance superior CD8 responses compared to all other modalities, tested as well as the induction of CD4 responses that were barely detectable in groups of mice immunised with other modalities.

### Example 11

### Mixed Modality Delivery Using NHP Adenovirus Delivered Intramuscularly Followed by PMED in Minipigs

The induction of immune responses in a large animal model, the minipig, using mixed modality delivery was measured. Groups of 5 minipigs received either 3×10¹¹, 3×10¹⁰, 3×10⁹ or 3×10⁸ particles of NHP Adv Pan 6 at prime and were boosted with RNG plasmid delivered by PMED 12 and 24 weeks later. For PMED, animals each received 4 cartridges delivered at non-overlapping sites on the ventral abdomen, each cartridge containing approximately 0.8 μg plasmid DNA. Immune responses were assessed by IFNγ ELISPOT on peripheral blood mononuclear cells collected at intervals after both prime and boost and restimulated in vitro using pools of peptide libraries to the HIV antigens.

Responses were detected in minipigs after the prime with NHP Adv at the two highest doses (ie. 3×10¹¹ and 3×10¹⁰ particles) and in all groups after the PMED boost, with the strength of response correlating with NHP Adv priming response (FIG. 16). The post boost responses in the groups that received the two highest doses greatly exceeded those obtained following single modality PMED prime and boost indicating considerable advantage of NHP Adv/PMED mixed modality over single modality in this large animal model.

### Example 12

### Construction of CPC-P501S Expression Plasmid

A gene encoding human P501S fused to CPC was constructed by overlapping PCR incorporating restriction sites to enable ligation into the DNA vaccine vector p7313 (details included in WO02/08435 and WO2003104272, the entirety of which earlier publication is incorporated herein by reference). The DNA and protein sequences of CPC-P501S are given in SEQ ID: 15 and SEQ ID: 16 respectively (see FIG. 20).

GM-CSF plasmid was prepared as set out in Example 3 above.

Co Delivery of Two Plasmids: p7313 Expressing CPC-P501S (Plasmid ID JNW773) and p7313 GMCSF Plasmids (Plasmid Encoding GM-CSF)

Plasmid DNA was precipitated onto 2 μm diameter gold beads using calcium chloride and spermidine. Equal amounts of plasmids encoding antigen CPC-P501S (JNW773) and p7313GMCSF plasmids were mixed and co-precipitated so that all beads were coated with a mixture of the 2 plasmids ensuring delivery of both plasmids to the same cell. Unless otherwise stated both the antigen and GMCSF were loaded at 1.0 μg/cartridge. Loaded beads were coated onto Tefzel tubing as described in, for example, Eisenbraum, et al. 1993. DNA Cell Biol. 12:791-797; Pertmer et al, 1996 J. Virol. 70:6119-6125). Particle bombardment was performed using the Accell gene delivery (PCT WO 95/19799; incorporated herein by reference). Female Balb/c mice were immunised with 2 administrations of plasmid at each time point as detailed in the results section, one on each side of the abdomen after shaving. The total dose of DNA at each time point was 4 μg. Where Imiquimod was delivered this was applied topically in a cream formulation over the immunisation site, 24 hours following immunisation. 20 μl of 5% Aldara™ cream (3M) was applied at each immunisation site.

### P501S-Adenovirus

The replication-deficient E11/E3 deleted adenovirus serotype 5 vector (Ad5-P501S) was constructed by Corixa Corp. The adenovirus was delivered intramuscularly at 5×10⁶ pfu in 50 μl of PBS

### Preparation of Mouse Splenocytes

Spleens were obtained from immunised mice at 7 or 14 days post immunisation or the time point indicated on the figures. Spleens were processed by grinding between glass slides to produce a cell suspension. Red blood cells were lysed by ammonium chloride treatment and debris was removed to leave a fine suspension of splenocytes. Cells were resuspended at a concentration of 4×10⁶/ml in RPMI complete media for use in ELISPOT assays.

Flow Cytometry to detect IFNγ and IL-2 Production from Murine T Cells in Response to Peptide or Protein Stimulation

5×10⁶ splenocytes were aliquoted per test tube, and spun to pellet. The supernatant was removed and samples vortexed to break up the pellet. 0.5 μg of anti-CD28+0.5 μg of anti-CD49d (Pharmingen) were added to each tube, and left to incubate at room temperature for 10 minutes. 1 ml of medium was added to appropriate tubes, which contained either medium alone, or medium with peptide or protein at the appropriate concentration. Samples were then incubated for an hour at 37° C. in a heated water bath. 10 μg/ml Brefeldin A was added to each tube and the incubation at 37° C. continued for a further 5 hours. The programmed water bath then returned to 6° C., and was maintained at that temperature overnight.

Samples were then stained with anti-mouse CD4 Pe-Cy5 (Pharmingen) and anti-mouse CD8 ECD. (Beckman Coulter) Samples were washed and 100 μl of Fixative was added from the “Whole blood lysing reagent” kit (Beckman Coulter) for 15 minutes at room temperature. After washing, 100 μl of permeabilisation reagent from the same kit was added to each sample with anti-IFNγ-PE (Pharmingen)+anti-IL-2-FITC (Immunotech). Samples were incubated at room temperature for 15 minutes, and washed. Samples were resuspended in 1.0 ml buffer, and analysed on the Flow Cytometer. A total of 500,000 cells were collected per sample and subsequently CD4 and CD8 cells were gated to determine the populations of cells secreting IFNγ and/or IL-2 in response to stimulus.

### Example 13

### Mixed Modality Delivery Using NHP Adenovirus Intramuscular or Intra-Dermal

The induction of immune responses after mixed modality Adenovirus/PMED was measured. Different routes of immunisation for delivery of the Adenovirus were compared (intra-muscular and intra-dermal). Groups of 4 mice were immunised at prime with 1×10⁹ particles of NHP-Adv (Pan6GRN) by either the intra-dermal or intramuscular route and with 1 g of DNA expressing HIV RT, Nef and Gag (p73iRNG) antigens by PMED at the boost immunisation. Immunisations were given with an interval of 42 days. Improved responses were observed against RT and Gag antigens when intramuscular Adv/PMED mixed modality was compared with intra-dermal Adv/PMED delivery. Intra-dermal delivery of Adv in this regimen enhanced up to 2 fold CD8+ and CD4+ cells producing INF in response to RT and up to 4 fold CD4+ cells producing IL2 in response to either RT or GAG. Responses against RT and Gag antigens are illustrated in FIG. 21 (INF) and FIG. 22 (IL2). Delivery of Adv by the intra-dermal route in the mixed modality Adv/PMED regime shows the ability to achieve superior CD8 and CD4 responses compared to intramuscular Adv delivery in this regime.

### Example 14

### Mixed Modality Delivery Using NHP Adenovirus Delivered Intra-Muscularly Followed by PMED in Primates

The induction of immune responses in primates, using mixed modality delivery as described in example 11, was measured. Groups of 5 cynomolgus monkeys received either 3×10¹¹, 3×10¹⁰, 3×10⁹ or 3×10⁸ particles of NHP Adv Pan 6 at prime and were boosted with RNG plasmid delivered by PMED between weeks 17 and 24. A second boost was given at week 39. For PMED, animals each received 8 cartridges delivered at non-overlapping sites on the ventral abdomen, each cartridge containing approximately 0.8 μg plasmid DNA. Immune responses were assessed by IFNγ ELISPOT on peripheral blood mononuclear cells collected at intervals after both prime and boost and restimulated in vitro using pools of peptide libraries to the HIV antigens.

Responses were detected in primates after the prime with NHP Adv at the two highest doses (ie. 3×10¹¹ and 3×10¹⁰ particles) and in all groups after the PMED boost (see FIGS. 23a and 23b, NB results not shown for doses 3×10¹⁰ and 3×10⁹ particles). Whilst the level of the post boost responses did not appear to be dependent on the NHP Adv priming dose in this species, the responses were more consistent post PMED compared with the weak and variable responses induced in primates following PMED single modality prime and boosting (results not shown).

### DESCRIPTION OF FIGURES

FIG. 1 shows SEQ ID No.2—the sequence of the expression cassette containing the OvaCyt gene, start and stop codons are in bold.

FIG. 2 shows SEQ ID No.2—the sequence of the expression cassette containing the OvaSec gene, start and stop codons are in bold.

FIGS. 3 to 8 show polynucleotide sequences, amino acid sequences and restriction maps for constructs described in Example 2.

FIG. 9 shows immune responses specific for OVA antigen measured day 6 post boost. Splenocytes from immunised mice with different doses of Adv5 vector and or PMED DNA expressing OVA antigen, were incubated with or without OVA specific CD8 peptide (SIINFEKL) for 6 h. After incubation cells were treated for IFNγ intracellular staining and surface markers.

FIG. 10 shows the kinetic of responses after mixed modality delivery and using different intervals between immunisations. Ovalbumin tetramers specific responses were monitored in the blood of immunised animals. Time of boost immunisations are indicated by vertical lines (day 21 and day 110).

FIG. 11 shows the comparison of kinetic of responses using different routes and sequence to deliver DNA with Adv. Ovalbumin tetramer CD8⁺ specific cells were monitored in blood from immunised animals. Red bars correspond to animals primed with Adv (5.106 pfu) and boosted with DNA (PMED, intra-muscular, intra-dermal). Blue bars correspond to animals primed with DNA and boosted with either Adv (three central bars) of DNA (three right-hand bars).

FIG. 12 shows cellular responses after the second boost immunisation as monitored by production of cytokines. Cellular responses CD8 (left panel) and CD4 (right panel) were monitored using OVA specific CD8 (SIINFEKL) and CD4 (TWETSSNVMEERKIKV) peptides. Intracellular cytokines were detected using anti mouse IFNγ and IL2 antibodies.

FIG. 13 shows Cellular responses induced after mixed modality delivery in combination with molecular adjuvant. Mice primed with Adv5-OVA (adeno) were boosted with DNA OVA (Ova cyt or Ova sec froms) adjuvantised (DNA GM-CSF+Aldara™ cream) or not adjuvantised. Day 7 after boost immune responses were monitored by tetramer specifics (spleen and blood—top panel) and cytokine production monitored by cytokine specific-intracellular staining (bottom panel).

FIG. 14 shows the cellular responses comparing mixed modality delivery and homologous delivery using NHP adenovirus vectors delivered intra-dermally (ID.) (FIG. 14(a)) and Intra-muscularly (IM) (FIG. 14(b)) Cellular immune responses were monitored after boost immunisation by flow cytometrie. Cellular responses CD8 (left panel) and CD4 (right panel) were monitored using HIV Gag specific CD8 (AMQMLKETI) and CD4 (YKRWIILGLNKIIR) peptides. Intracellular cytokines were detected using anti mouse IFNγ and IL2 antibodies.

FIG. 15 shows the cellular responses induced after heterologous Prime/boost delivery using, NHP adenovirus vectors (intra-dermal) Immune responses after boost. Cellular responses CD8 (left panel) and CD4 (right panel) were monitored using HIV Gag specific CD8 (AMQMLKETI) and CD4 (YKRWIILGLNKIIR) peptides. Intracellular cytokines were detected using anti mouse IFNγ and IL2 antibodies.

FIG. 16 shows the cellular response induced after mixed modality NHPAdv/PMED in minipigs. Minipigs were primed with NHP Adv and boosted with RNG plasmid by PMED and peripheral blood lymphocytes were monitored for immune responses by IFNγ ELISPOT. FIG. 16a shows the responses to a range of NHP Adv doses post prime and post first boost. FIG. 16b shows the responses to a range of NHP Adv doses post prime and post first and second PMED boost. FIG. 16c shows the comparison between mixed modality (NHP Adv prime and PMED boost) and single modality (PMED prime and PMED boost).

FIG. 17 shows the total percentage of CD8 cells expressing IFN-gamma at day 7 post-boost immunisation as determined by ICS (intracellular cytokine staining). Groups of 3-5 mice were immunised with either 5×10⁶ pfu's of a Human Ad5 expressing P501S (A) or with P501S DNA (D) by PMED. Each DNA immunisation (D) consists of 2×2 μg of a 1:1 mixture of a plasmid expressing CPC-P501S (plasmid ID=JNW773) and mouse GM-CSF. For all DNA immunisations by PMED, Aldara™ cream was applied 24 hours post-immunisation topically at the site of immunisation. Mice were immunised at day 0 and day 42.

FIG. 18 shows the total percentage of CD8 cells expressing IFN-gamma at day 7 post-31d immunisation as determined by ICS (intracellular cytokine staining). Groups of 3-5 mice were immunised with either 5×10⁶ pfu's of a Human Ad5 expressing P501S (A) or with P501S DNA (D) by PMED or with empty DNA (E) by PMED. Each DNA immunisation (D) consists of 2×2 μg of a 1:1 mixture of a plasmid expressing CPC-P501S (plasmid ID=JNW773) and mouse GM-CSF. Each empty DNA immunisation (E) consists of 2×2 μg of a 1:1 mixture of empty plasmid (p7313-ie) and mouse GM-CSF. For all DNA immunisations by PMED, Aldara™ cream was applied 24 hours post-immunisation topically at the site of immunisation. Mice were immunised at day 0, day 42 and day 84.

FIG. 19 shows the total percentage of CD8 cells expressing IFN-gamma at day 7 and day 14 post-4^{th} immunisation as determined by ICS (intracellular cytokine staining). Groups of 3-5 mice were immunised with either 5×10⁶ pfu's of a Human Ad5 expressing P501S (A) or with P501S DNA (D) by PMED or with empty DNA (E) by PMED. Each DNA immunisation (D) consists of 2×2 μg of a 1:1 mixture of a plasmid expressing CPC-P501S (plasmid ID=JNW773) and mouse GM-CSF. Each empty DNA immunisation (E) consists of 2×2 μg of a 1:1 mixture of empty plasmid (p7313-ie) and mouse GM-CSF. For all DNA immunisations by PMED, Aldara™ cream was applied 24 hours post-immunisation topically at the site of immunisation. Mice were immunised at day 0, day 42, day 84 and day 126.

FIG. 20 shows SEQ ID No. 15 and 16: DNA and protein coding sequence of CPC-P501S from plasmid JNW773

FIG. 21 shows elispot data indicating levels of CD4 and CD8 cells expressing IFN-gamma in response to RT, 21 days post-boost of mice primed with adenovirus (intra-dermal or intra-muscular) and boosted with DNA delivered by PMED.

FIG. 22 shows elispot data indicating levels of CD4 and CD8 cells expressing IL2 in response to RT, 21 days post-boost of mice primed with adenovirus (intra-dermal or intra-muscular) and boosted with DNA delivered by PMED.

FIG. 23 shows elispot data indicating levels of cells expressing IFNγ in response to RT, Gag and Nef, after priming with NHP Adv and after two PMED boosts. Results are shown for two doses of NHP Adv: 3×10¹¹ (FIG. 23a) and 3×10⁸ particles (FIG. 23b).

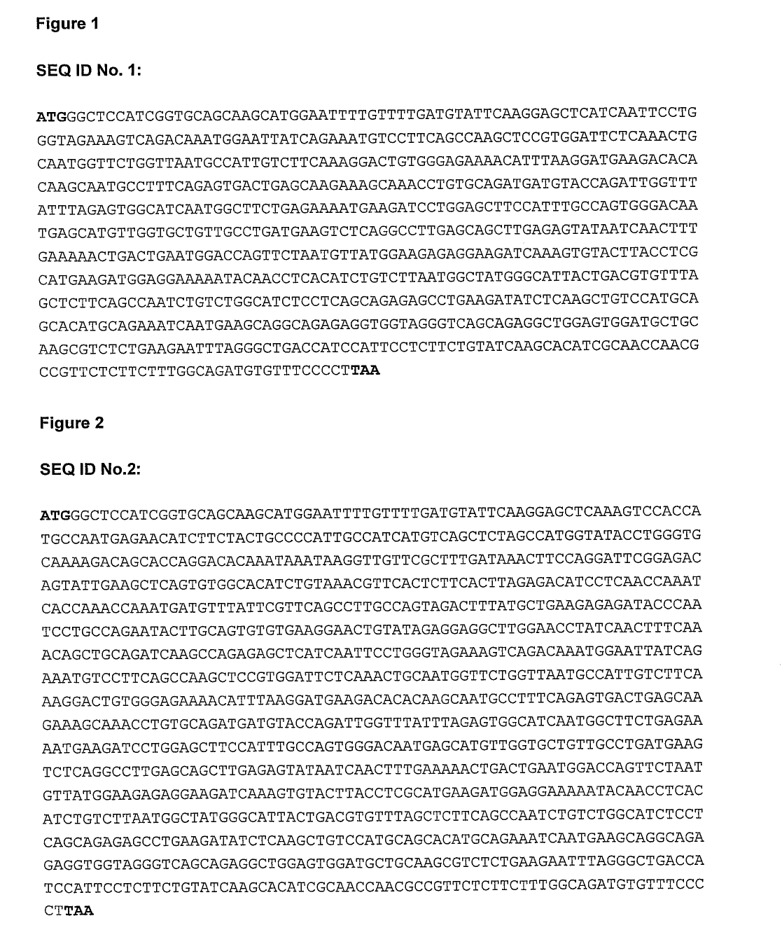

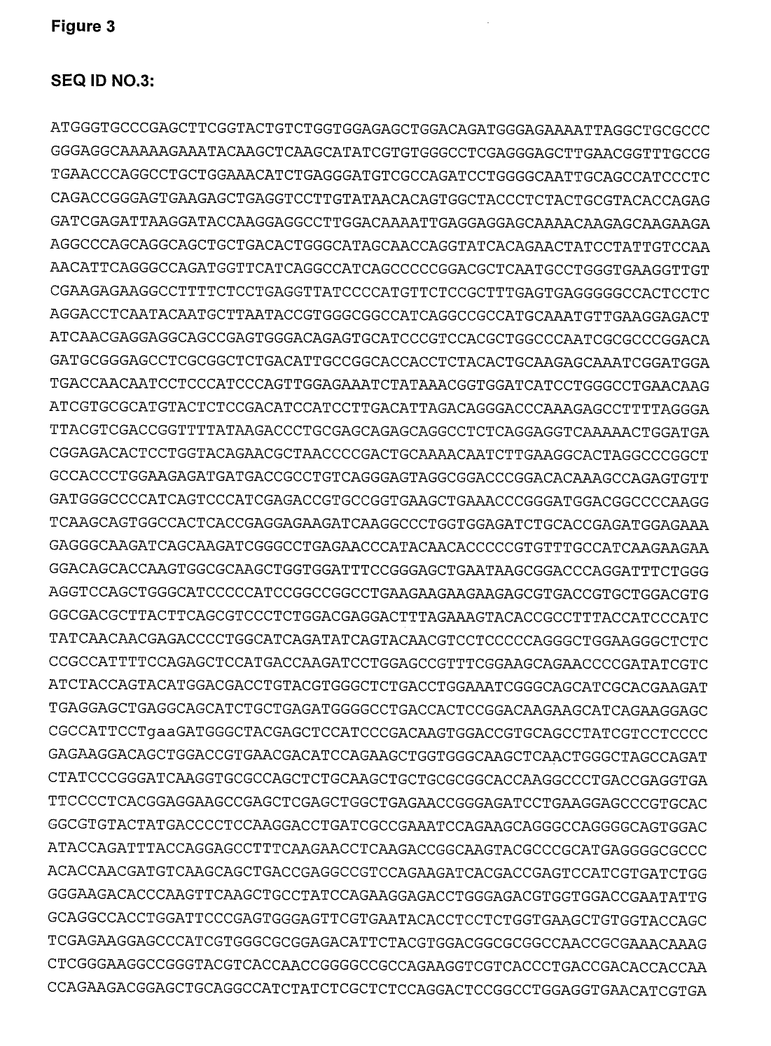

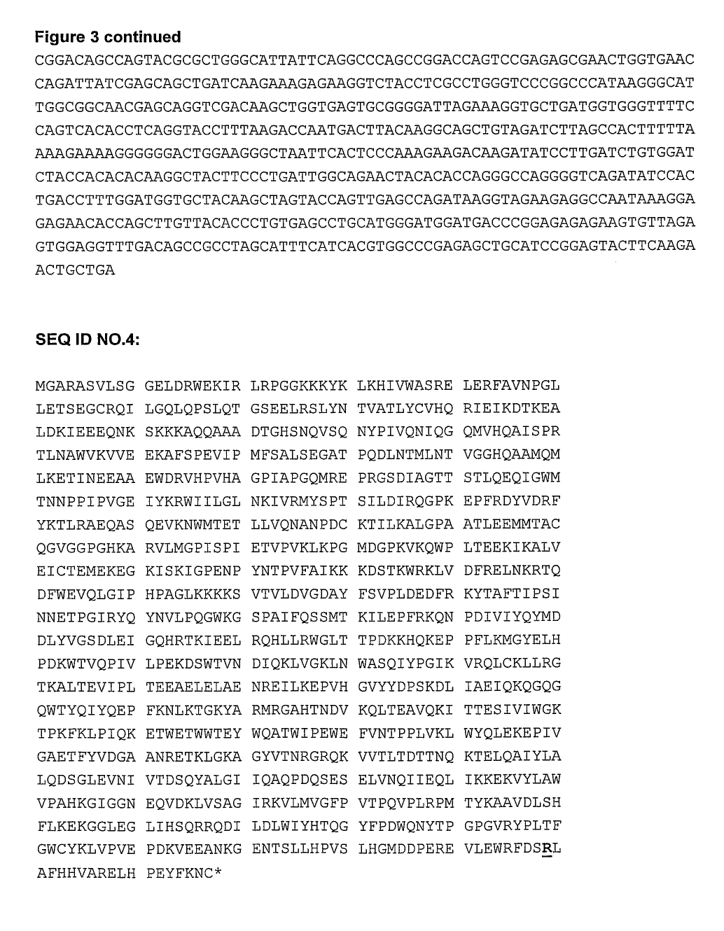

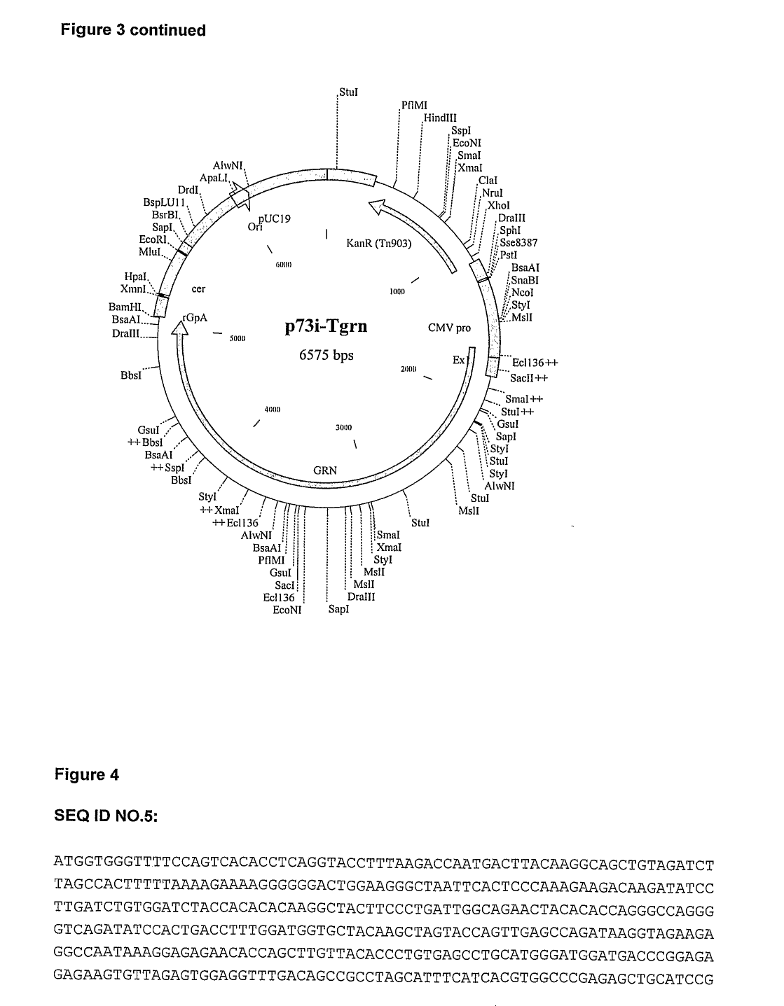

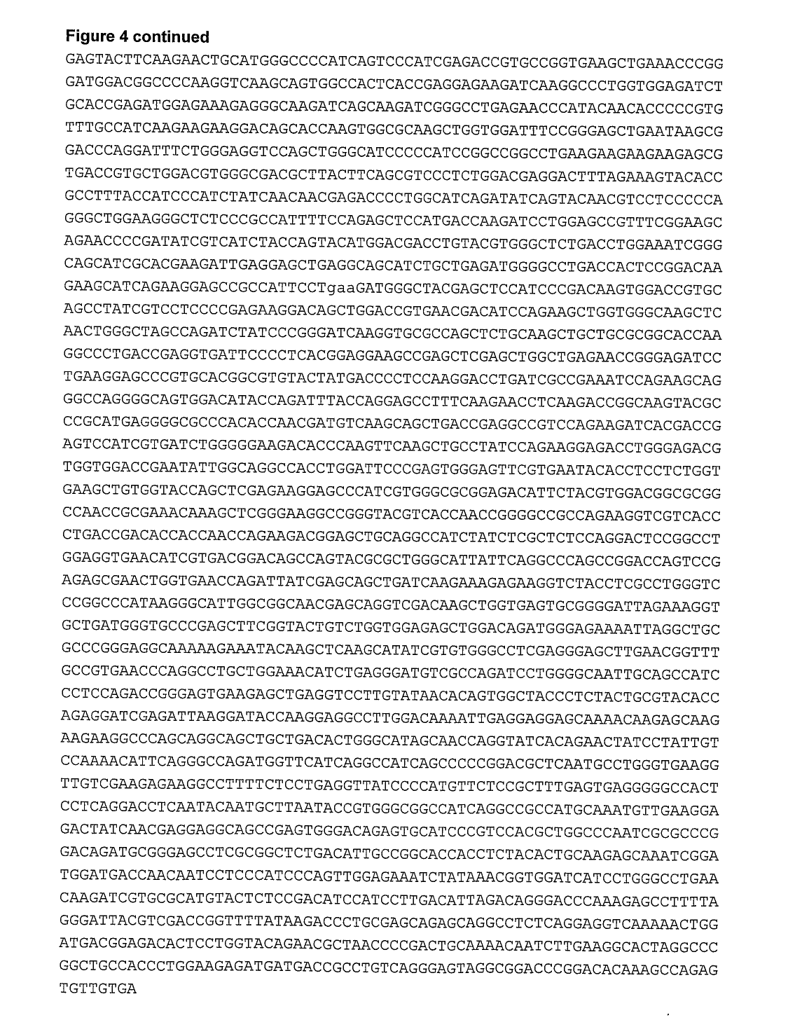

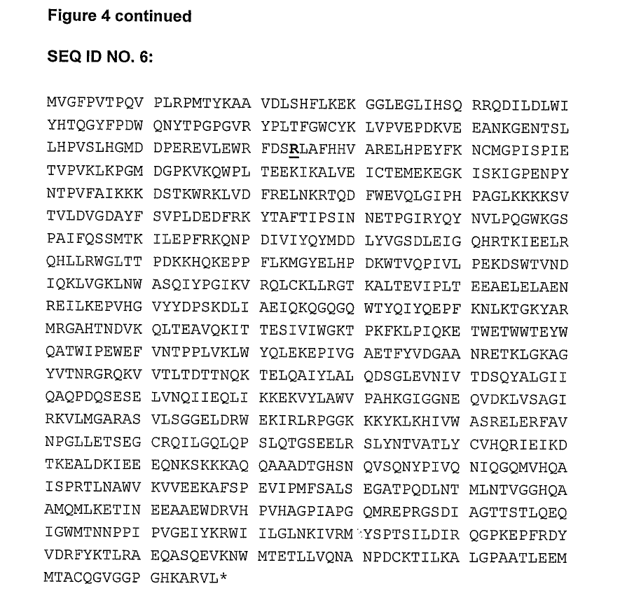

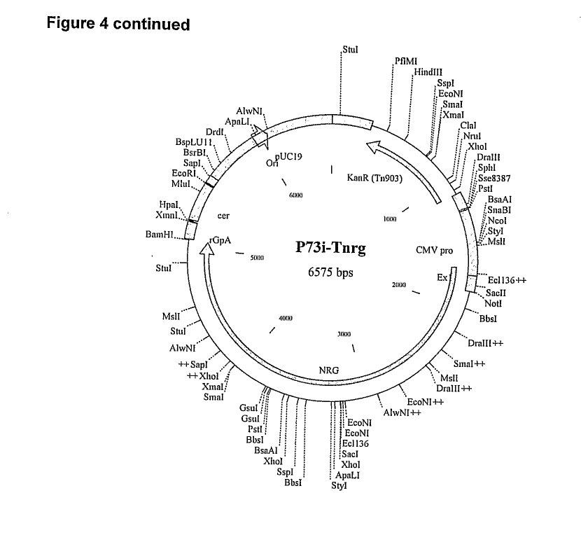

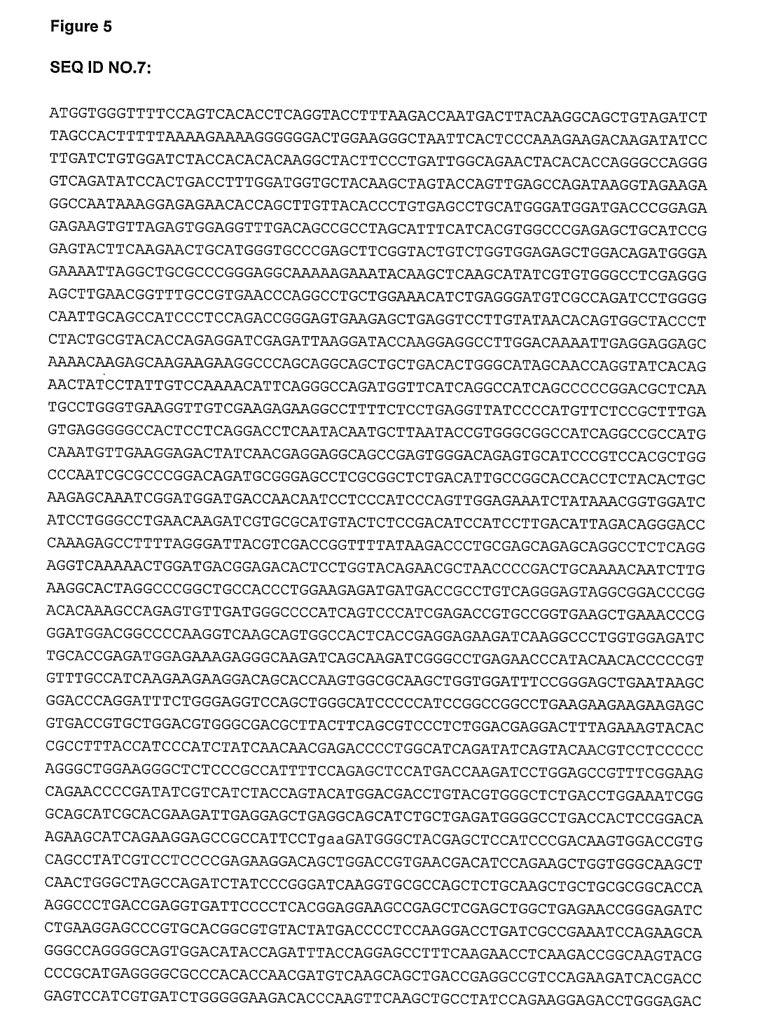

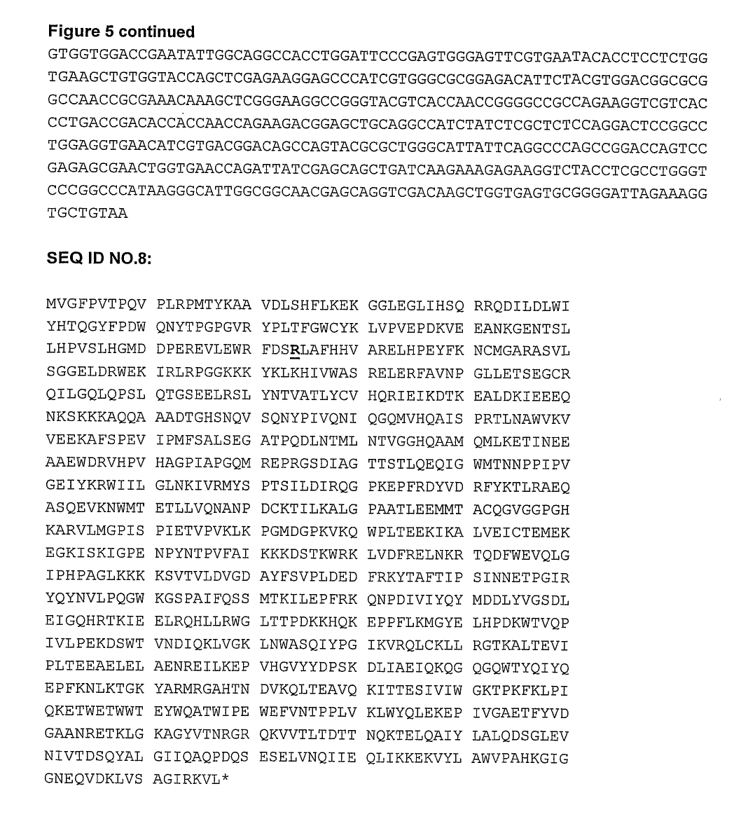

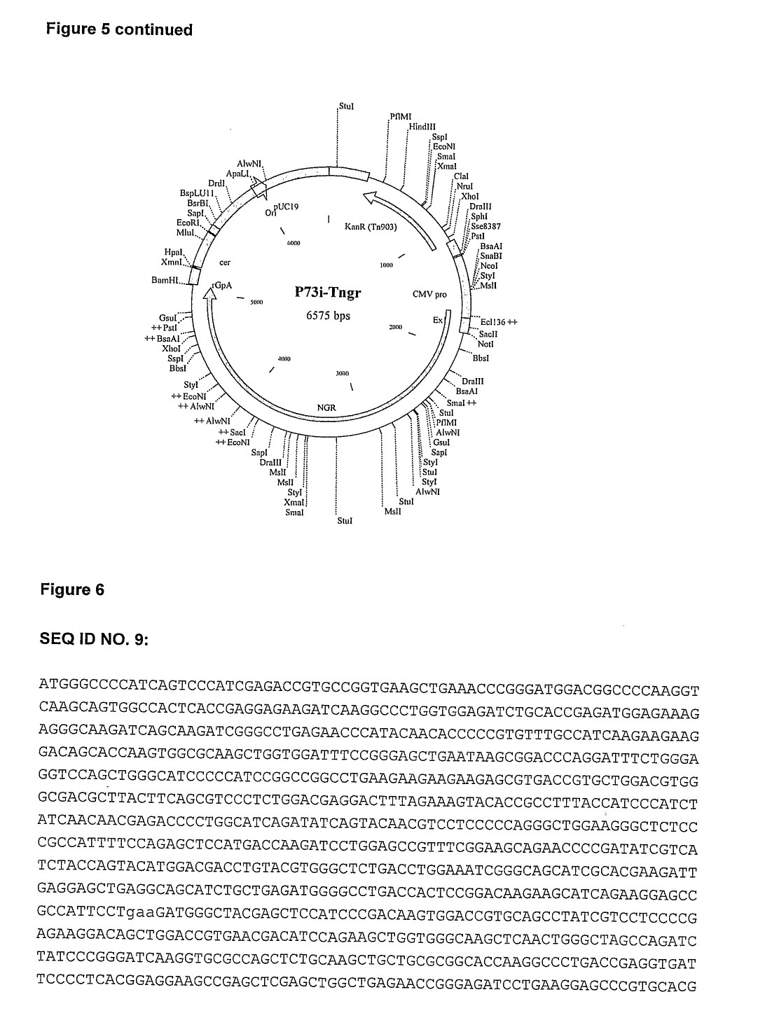

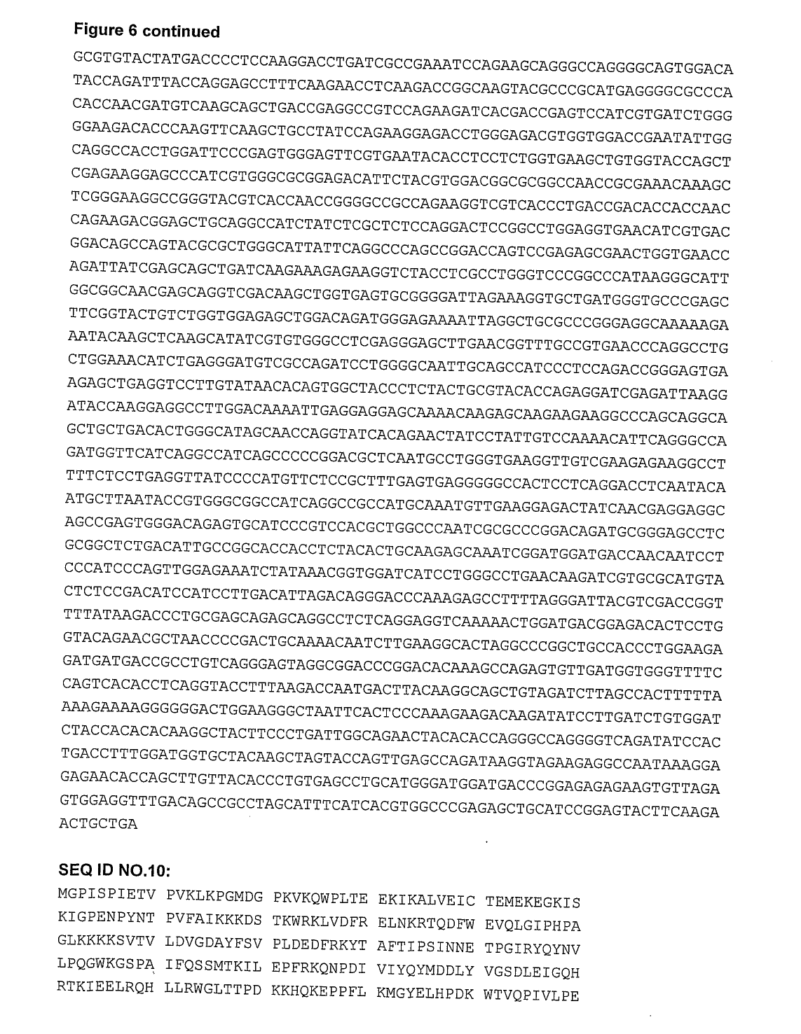

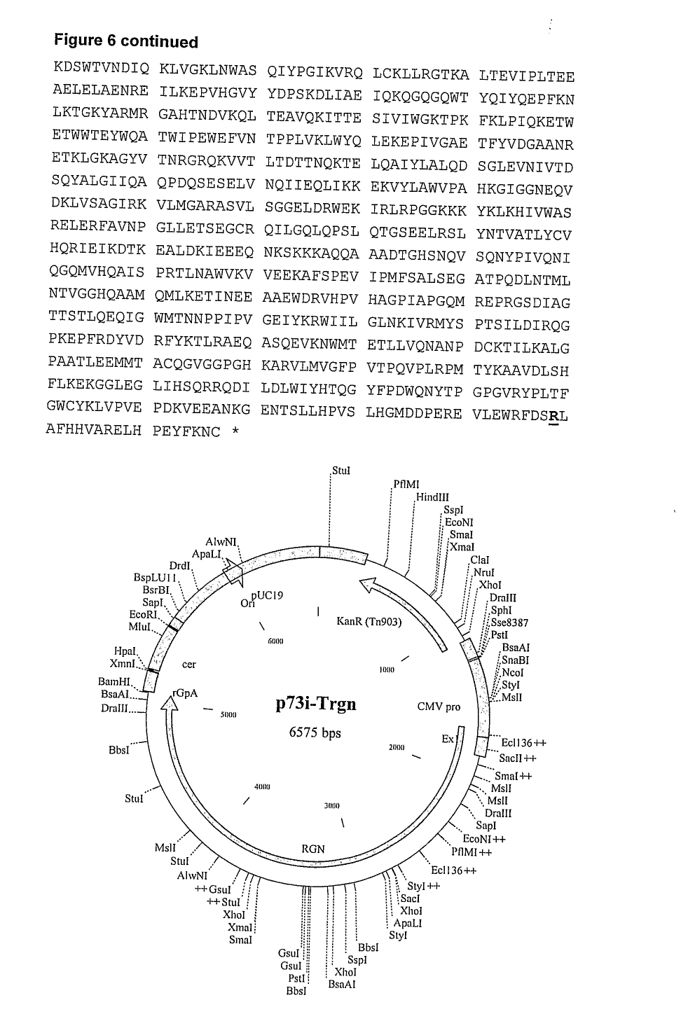

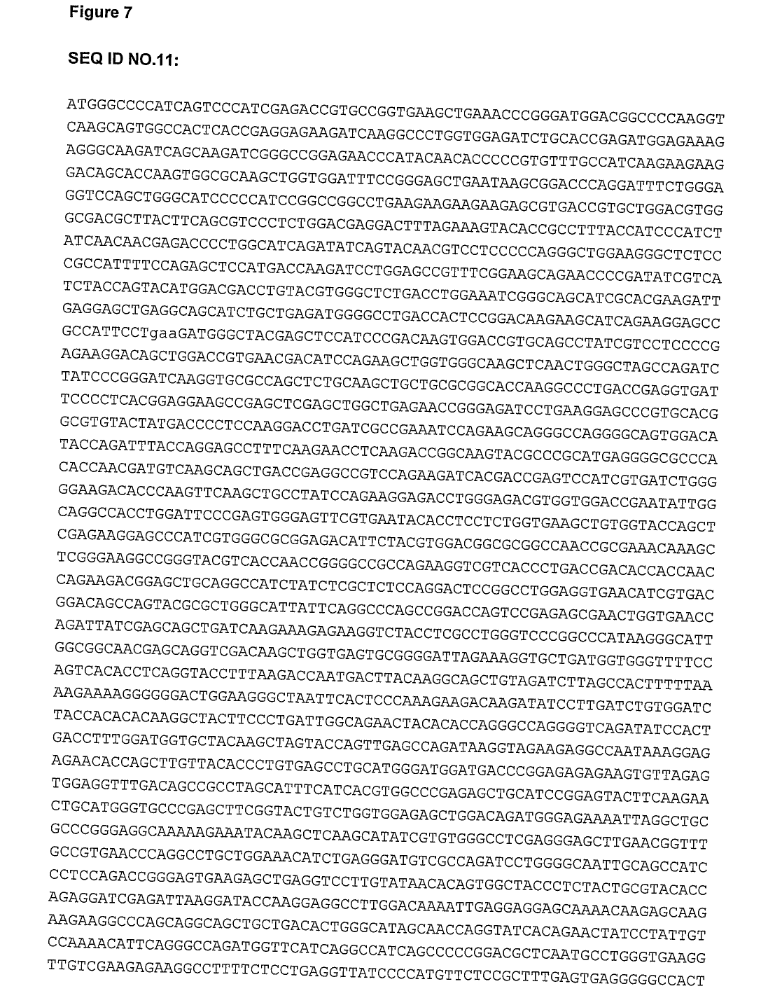

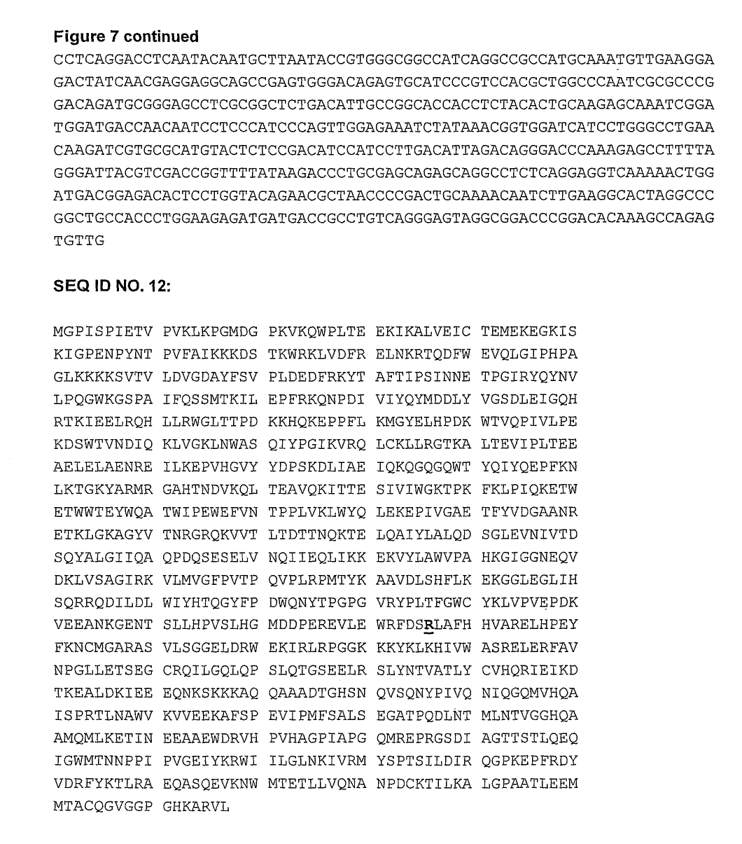

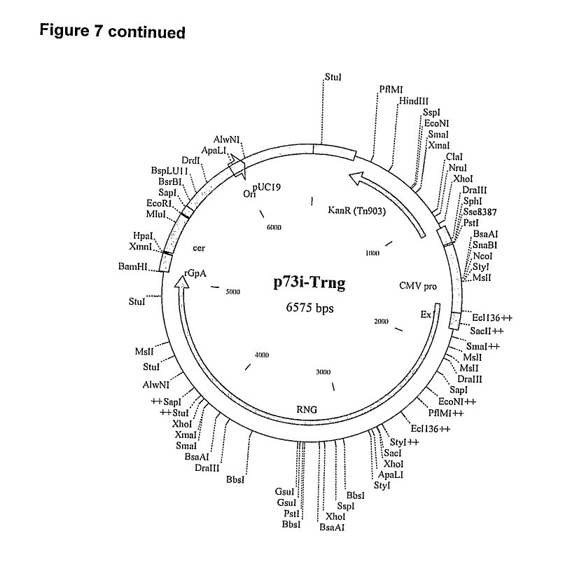

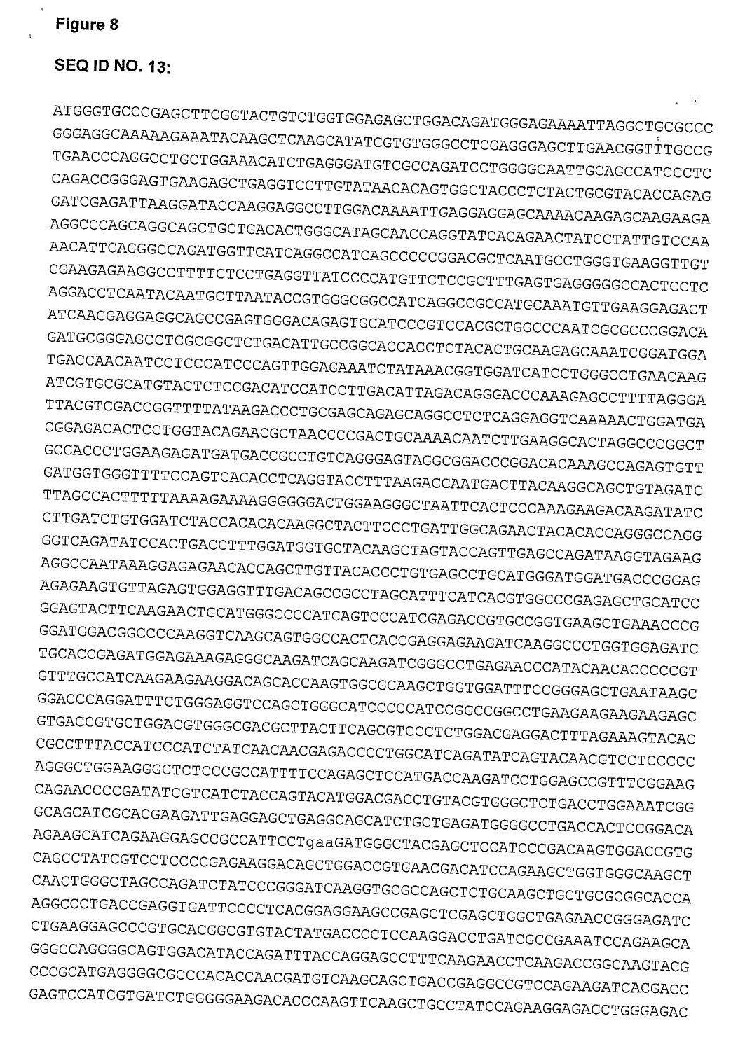

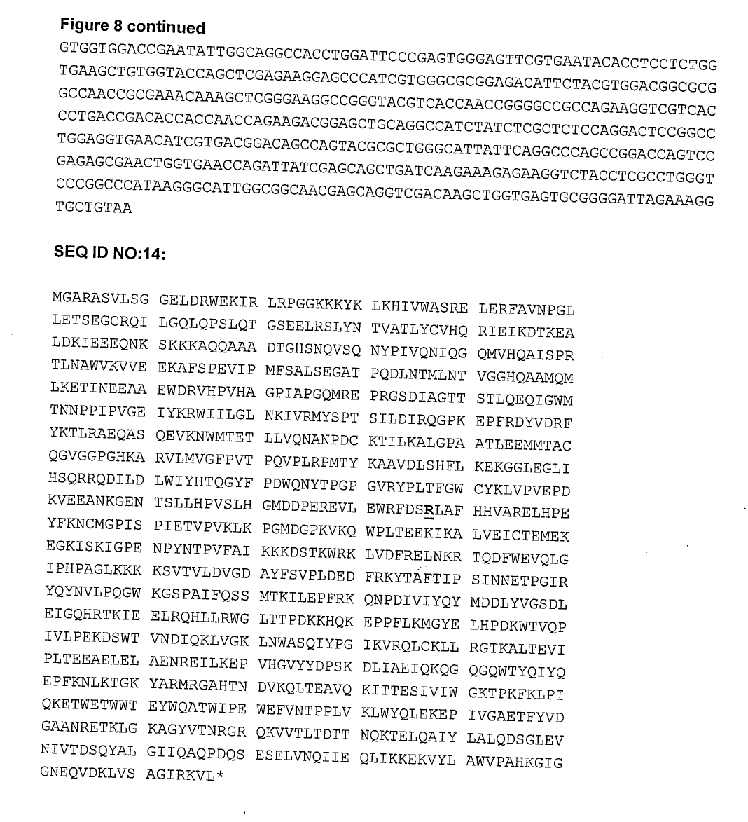

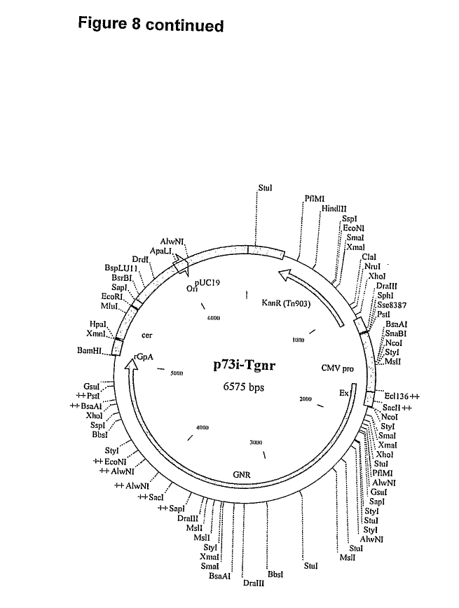

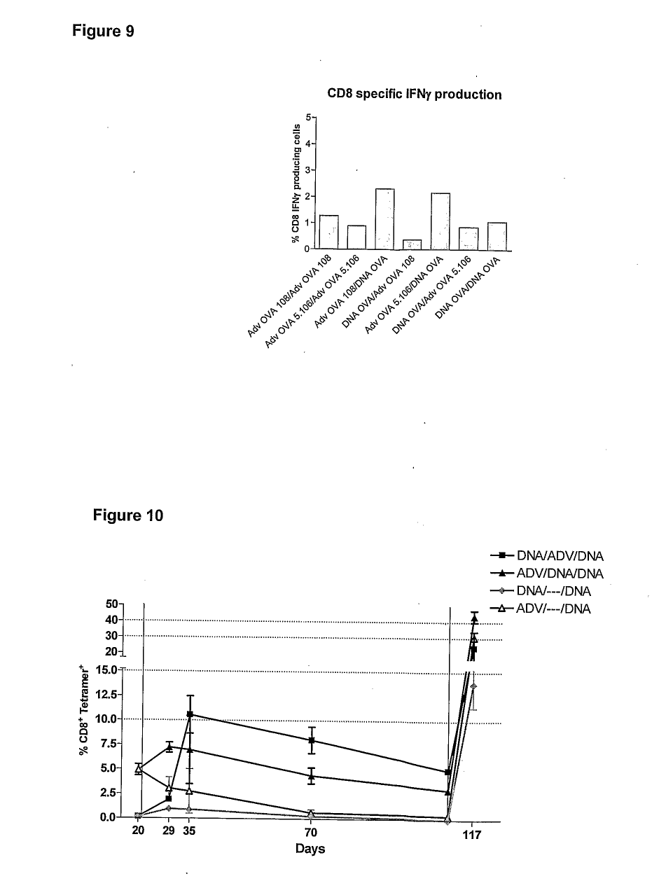

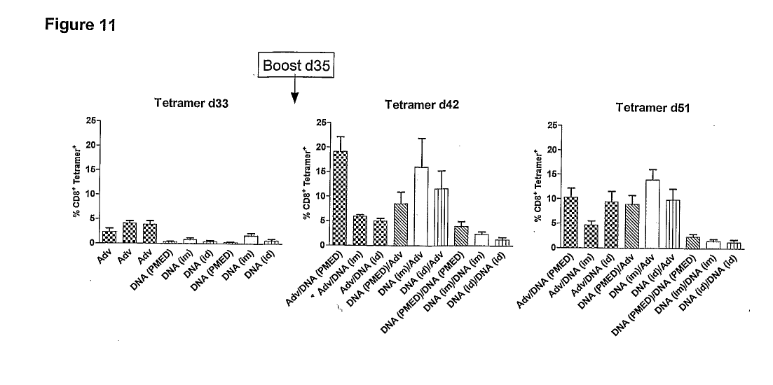

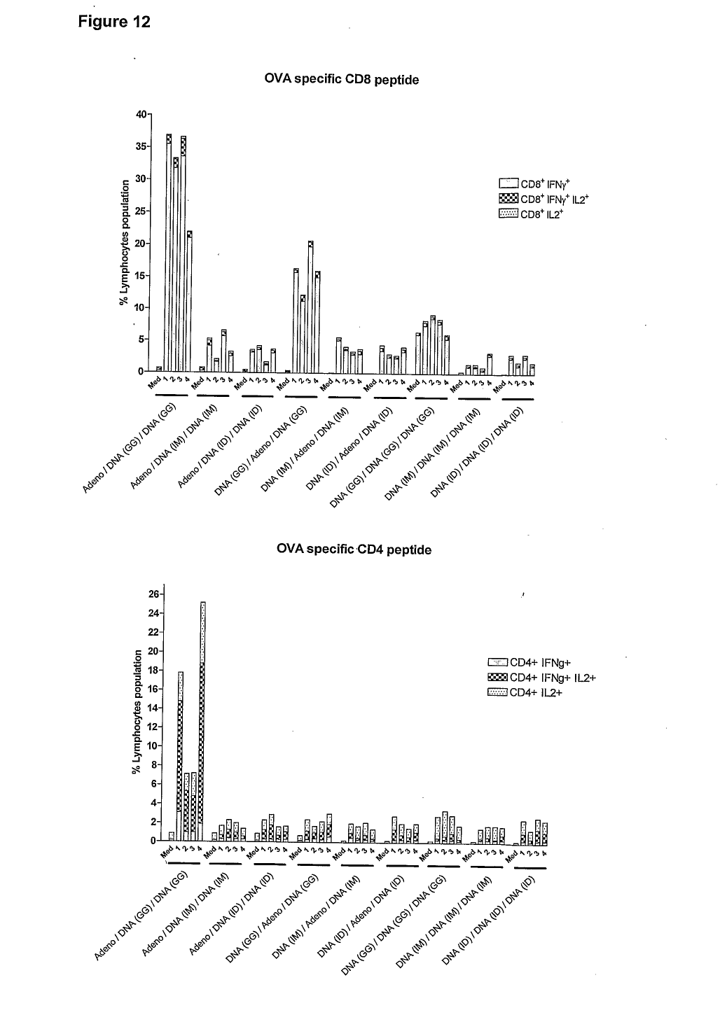

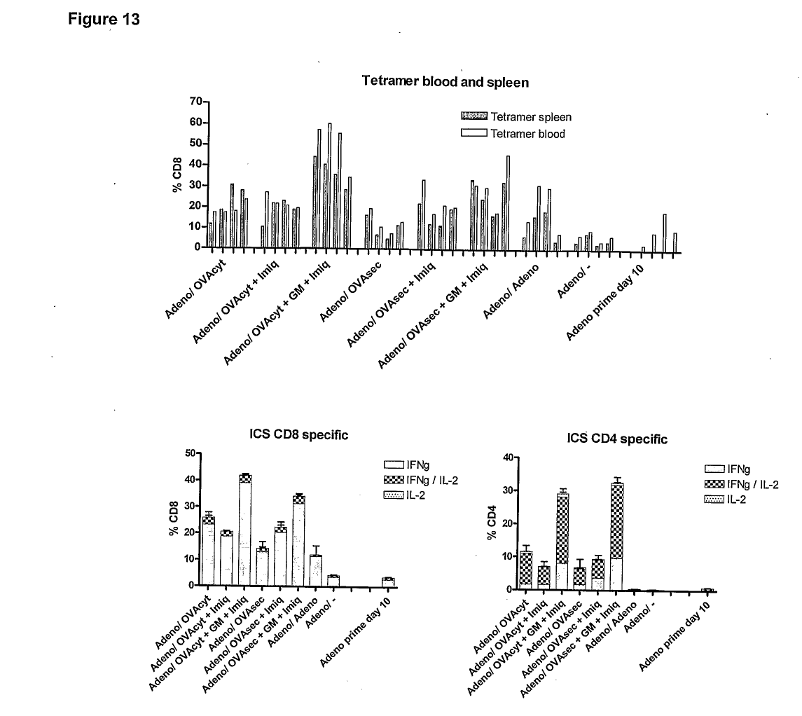

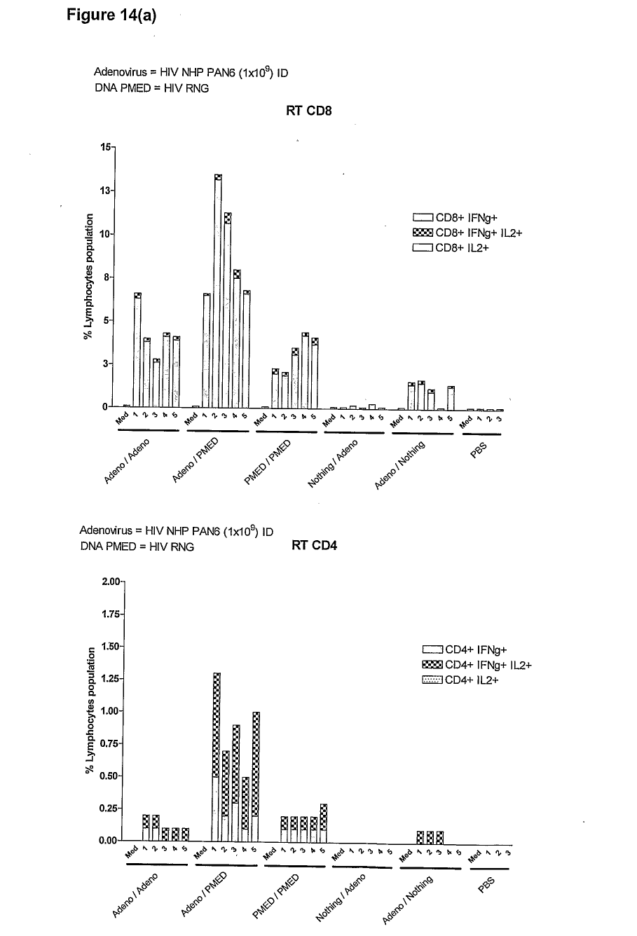

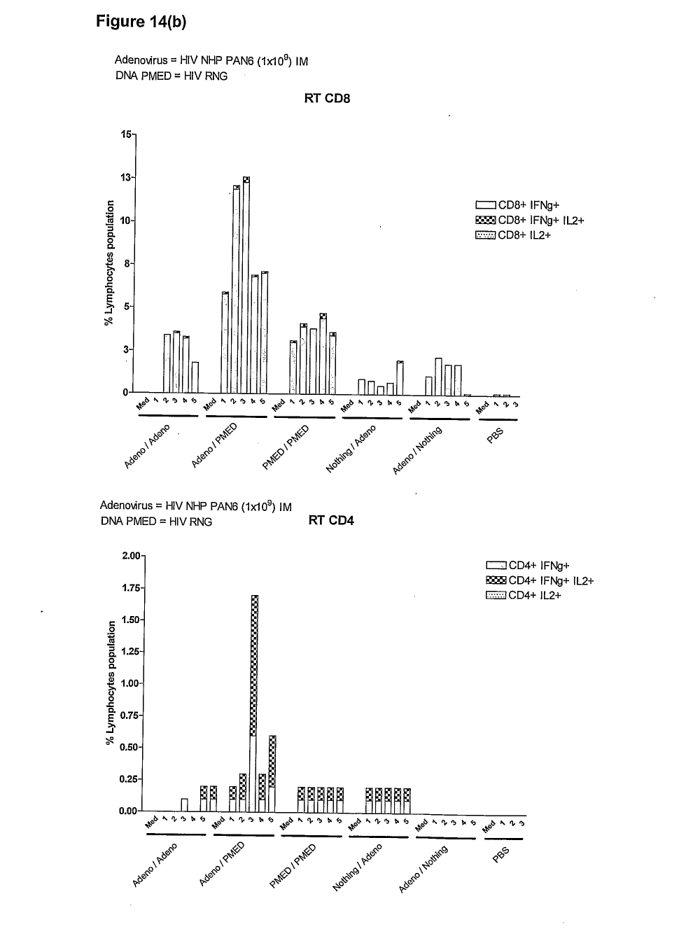

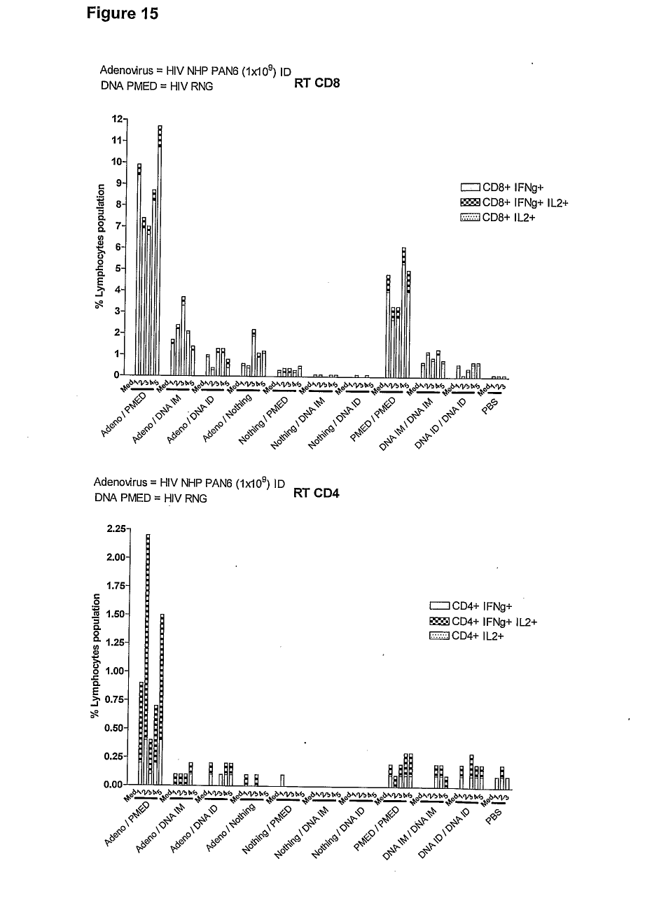

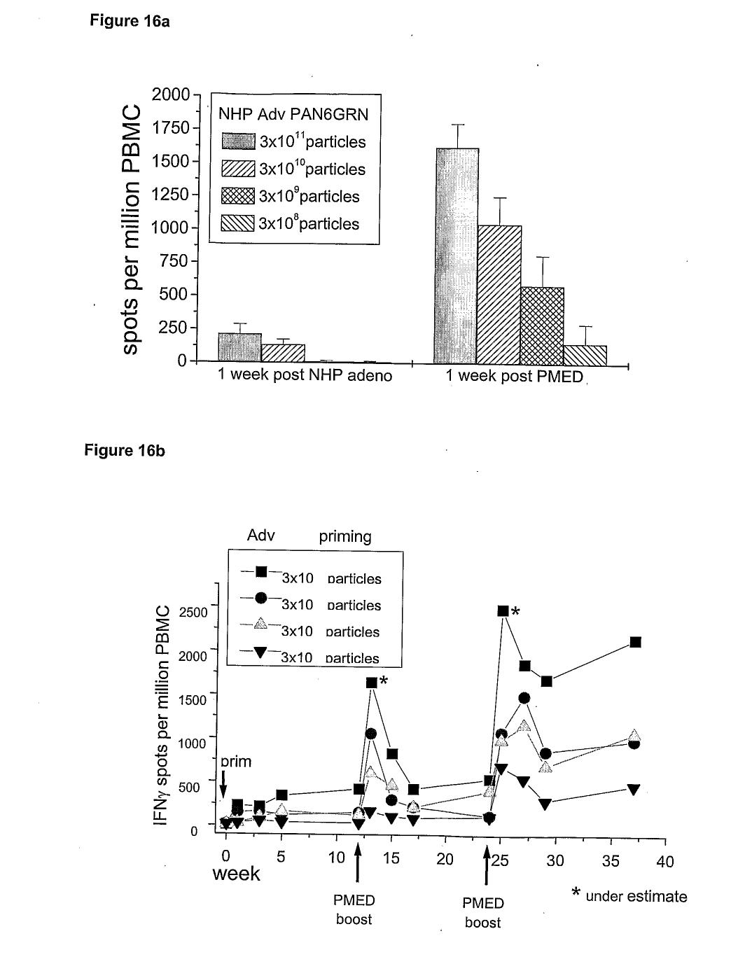

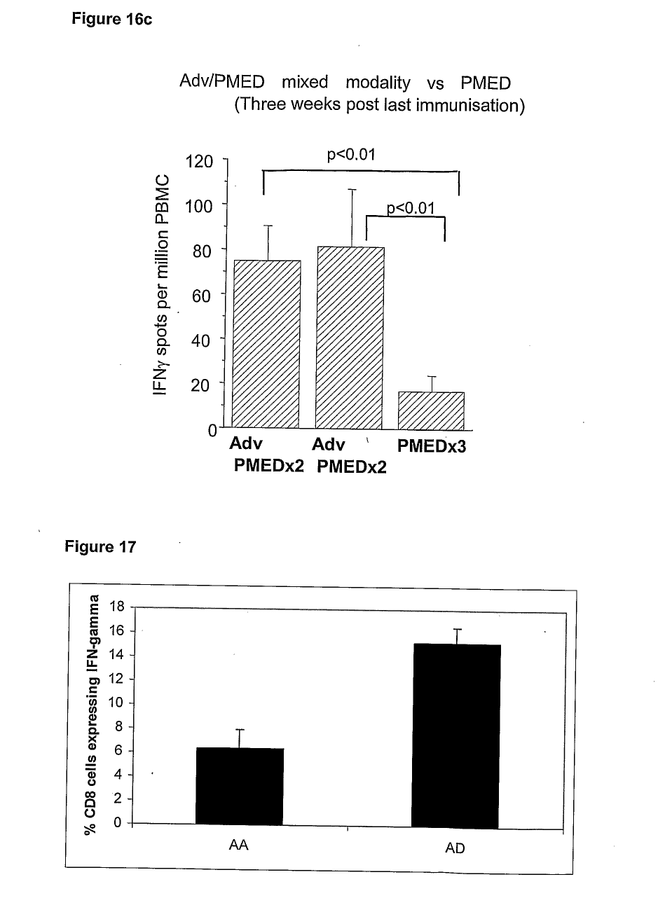

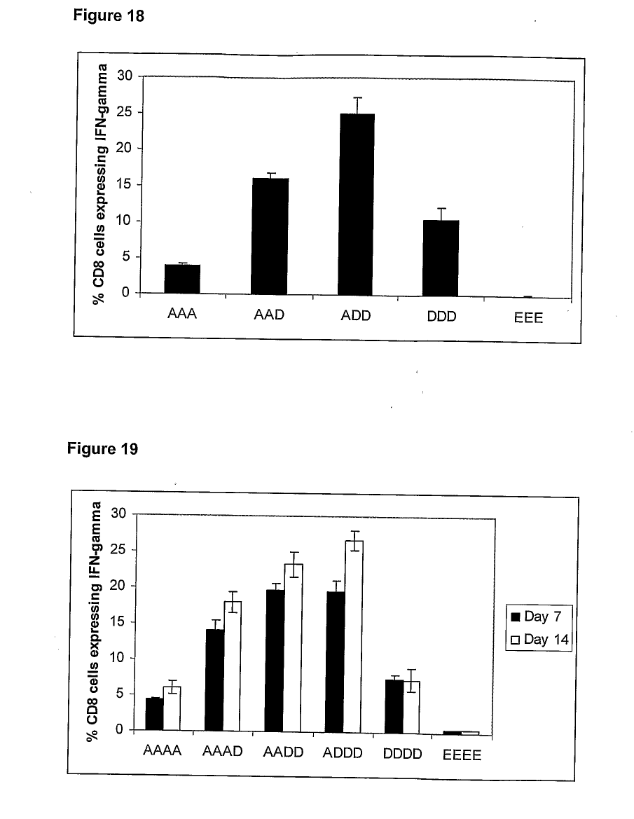

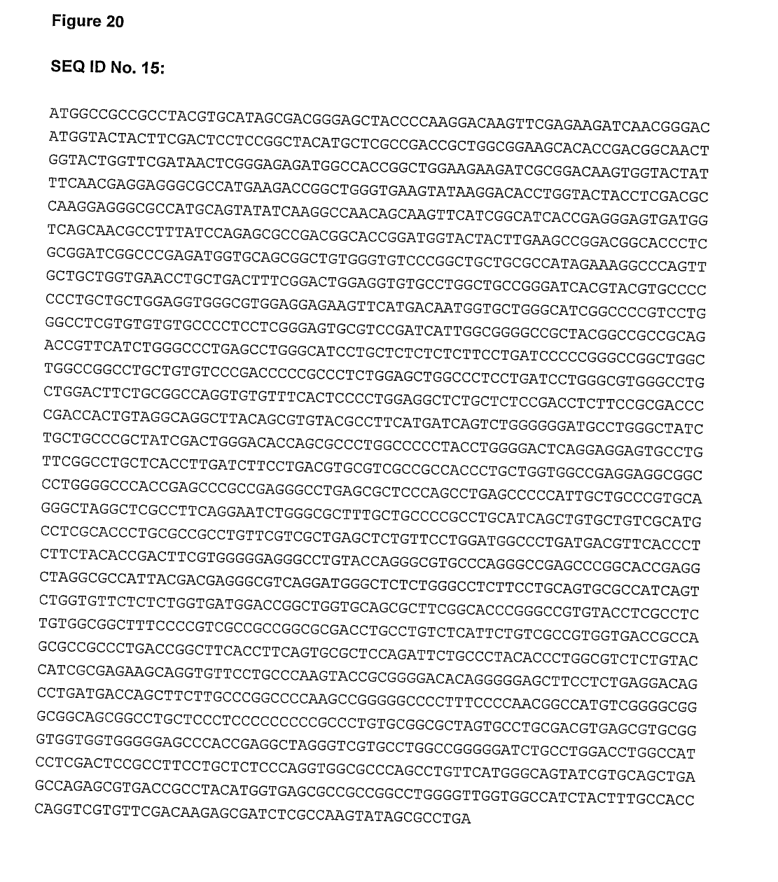

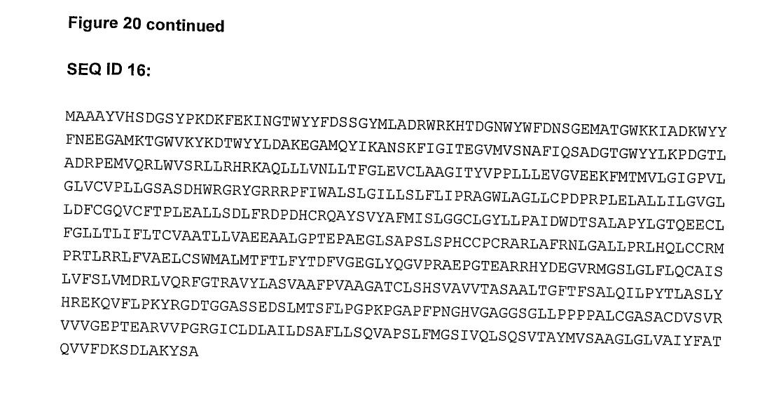

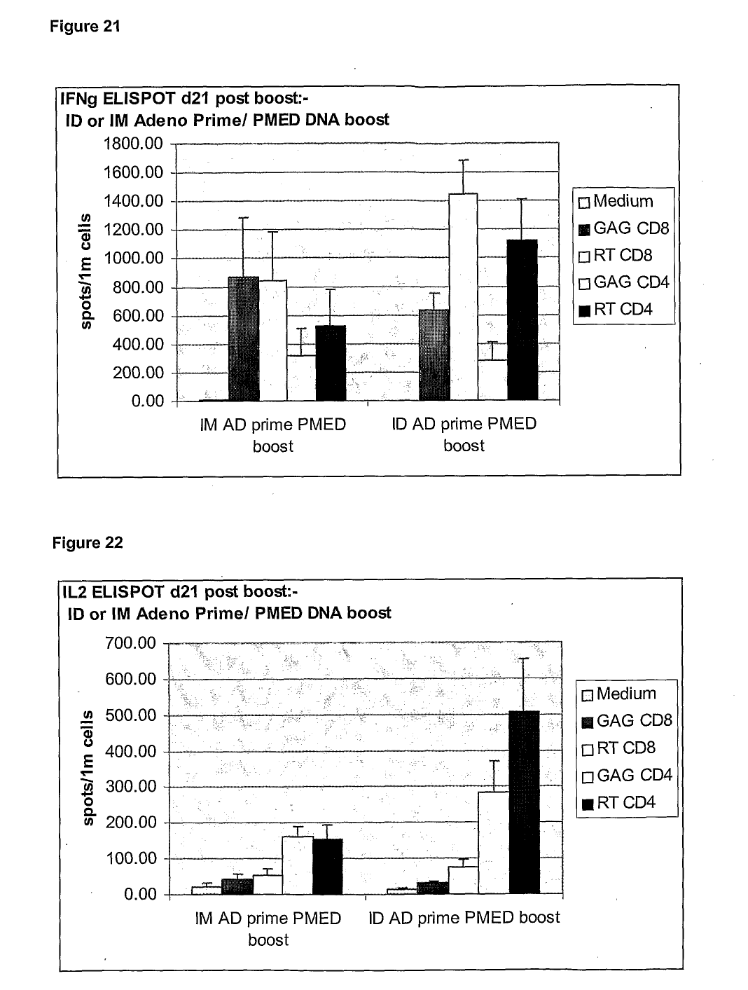

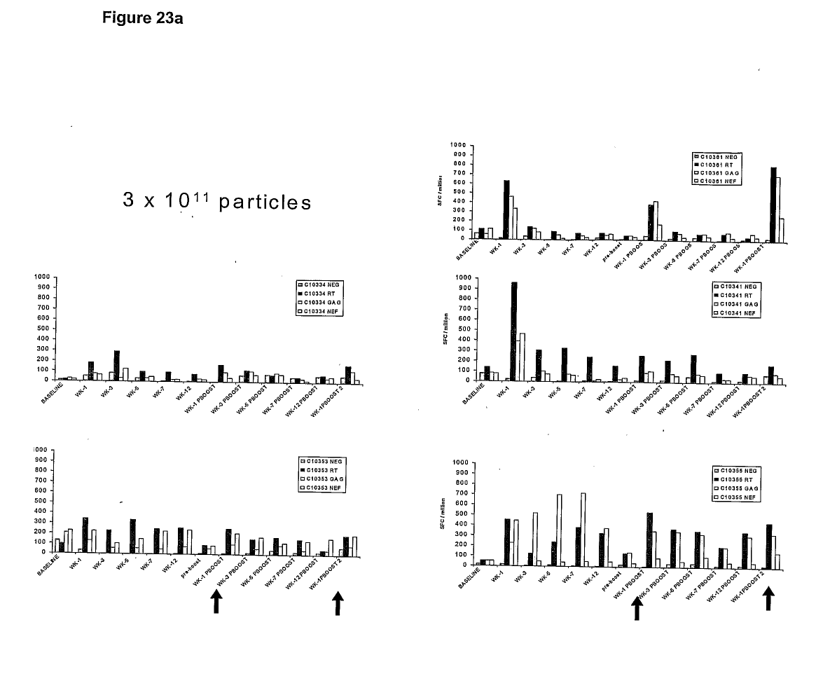

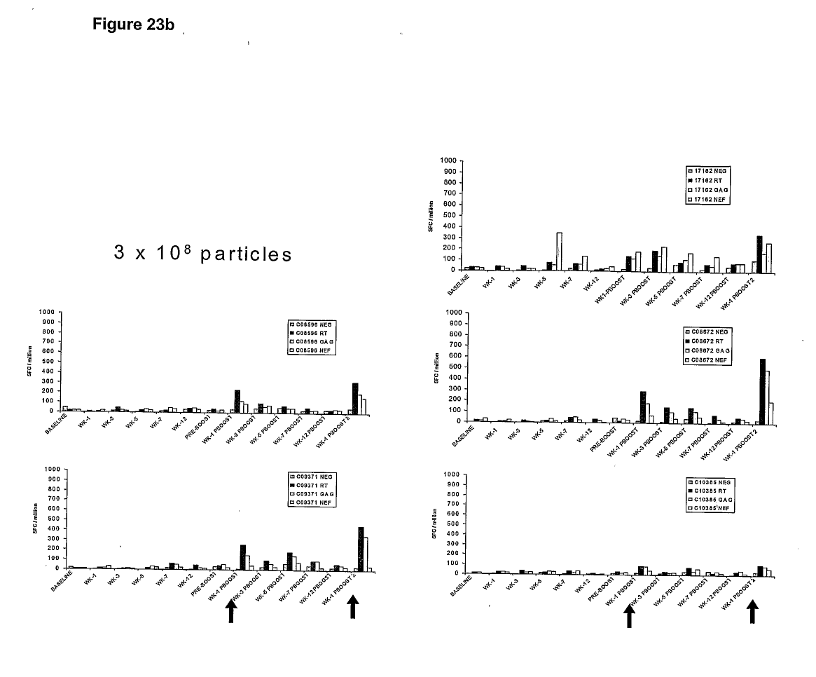

TABLE 1

| Amino acid or | Sequence Identifier |
|---|---|
| polynucleotide description | (SEQ ID No) |
| Ovacyt polynucleotide | 1 |
| Ovasec polynucleotide | 2 |
| Tgrn polynucleotide | 3 |
| Tgrn amino acid | 4 |
| Tnrg polynucleotide | 5 |
| Tnrg amino acid | 6 |
| Tngr polynucleotide | 7 |
| Tngr amino acid | 8 |
| Trgn polynucleotide | 9 |
| Trgn amino acid | 10 |
| Trng polynucleotide | 11 |
| Trng amino acid | 12 |
| Tgnr polynucleotide | 13 |
| Tgnr amino acid | 14 |
| CPC-P501S polynucleotide | 15 |
| CPC-P501S polypeptide | 16 |

1. Method of eliciting an immune response in a mammalian subject by administration of an adenoviral vector comprising a polynucleotide encoding a heterologous first non-self antigen, and a subsequent administration of a polynucleotide encoding a heterologous second non-self antigen comprising at least one epitope of the first heterologous non-self antigen, characterised in that the polynucleotide encoding the second heterologous non-self antigen is coated on or incorporated in a particle, and the particle is administered to the subject by a particle acceleration device.

2. A method according to claim 1 wherein the polynucleotide encoding the second heterologous non-self antigen is administered at least 7 days after the polynucleotide encoding the heterologous first non-self antigen is administered.

3. A method according to claim 1 wherein the immune response is a protective immune response.

4. A method according to claim 1 wherein the immune response is a therapeutically effective immune response.

5. A method according to claim 1 wherein the epitope is a T-cell epitope.

6. A method according to claim 1 wherein the heterologous non-self antigen is selected from one or more Nef, Gag, RT, Pol, Env, or immunogenic fragments or immunogenic derivatives thereof.

7. A method according to claim 1 wherein one or more adjuvants or polynucleotides encoding one or more adjuvants is co-administered with the heterologous non-self antigen.

8. A method according to claim 7 wherein the adjuvant is selected from imiquimod and GM-CSF.

9. A method according to claim 1 wherein the adenoviral vector is derived from a non-human primate adenovirus.

10. A method according to claim 9 wherein the non-human primate adenovirus is selected from Pan 5, Pan 6, Pan 7 and Pan 9.

11. A method according to claim 1 wherein the subject is human.

12. A kit comprising:
(i) a first vaccine comprising an adenoviral vector comprising a polynucleotide encoding a heterologous non-self antigen capable of raising an immune response
(ii) a second vaccine comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device.

13. A kit comprising:
(i) a first composition comprising an adenoviral vector comprising a polynucleotide encoding a heterologous non-self antigen capable of raising an immune response, and
(ii) a second composition comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device for use in medicine.

14. Method of treating HIV comprising administering to a mammalian subject a first composition comprising an adenoviral vector comprising a polynucleotide encoding a heterologous non-self antigen capable of raising an immune response and a second composition comprising a polynucleotide encoding a heterologous non-self antigen comprising at least one epitope of the first heterologous non-self antigen, characterised in that the polynucleotide encoding the second heterologous non-self antigen is coated on or incorporated in a particle and is formulated for delivery by a particle acceleration device.

15. The method of claim 14 wherein said mammalian subject is a human.
